SEQUENCE LISTING (138 sequences)

SEQ ID NO: 1 - DNA, 495 bp, Homo sapiens
  CDS (1)..(492)
atggcttcgccacaccaagagcccaaacctggagacctgattgagattttccgccttggctatgagcactgggccctgtatataggagatggctacgtgatccatctggctcctccaagtgagtaccccggggctggctcctccagtgtcttctcagtcctgagcaacagtgcagaggtgaaacgggagcgcctggaagatgtggtgggaggctgttgctatcgggtcaacaacagcttggaccatgagtaccaaccacggcccgtggaggtgatcatcagttctgcgaaggagatggttggtcagaagatgaagtacagtattgtgagcaggaactgtgagcactttgtcacccagctgagatatggcaagtcccgctgtaaacaggtggaaaaggccaaggttgaagtcggtgtggccacggcgcttggaatcctggttgttgctggatgctcttttgcgattaggagataccaaaaaaaagcgacagcctga

SEQ ID NO: 2 - protein, 164 aa, Homo sapiens
MASPHQEPKPGDLIEIFRLGYEHWALYIGDGYVIHLAPPSEYPGAGSSSVFSVLSNSAEVKRERLEDVVGGCCYRVNNSLDHEYQPRPVEVIISSAKEMVGQKMKYSIVSRNCEHFVTQLRYGKSRCKQVEKAKVEVGVATALGILVVAGCSFAIRRYQKKATA

SEQ ID NO: 3 - DNA, 19 bp, Artificial
  Synthetic nucleic acid
ggtaggctacgggtgaaac

SEQ ID NO: 4 - DNA, 20 bp, Artificial
  Synthetic nucleic acid
aacaactcaccaatatccgc

SEQ ID NO: 5 - DNA, 20 bp, Artificial
  Synthetic nucleic acid
gattttccgccttggctatg

SEQ ID NO: 6 - DNA, 20 bp, Artificial
  Synthetic nucleic acid
ttgctcaggactgagaagac

SEQ ID NO: 7 - DNA, 20 bp, Artificial
  Synthetic nucleic acid
gtggatgcagcatataagcc

SEQ ID NO: 8 - DNA, 20 bp, Artificial
  Synthetic nucleic acid
agtgaccaggtagatgacac

SEQ ID NO: 9 - DNA, 21 bp, Artificial
  Synthetic nucleic acid
ctattgctcccaggctacgca

SEQ ID NO: 10 - DNA, 22 bp, Artificial
  Synthetic nucleic acid
ccctgcaagctccattcgtagt

SEQ ID NO: 11 - DNA, 20 bp, Artificial
  Synthetic nucleic acid
cgcacaggaaaatcctacct

SEQ ID NO: 12 - DNA, 20 bp, Artificial
  Synthetic nucleic acid
acacaccacatccagattcc

SEQ ID NO: 13 - DNA, 21 bp, Artificial
  Synthetic nucleic acid
gggcctcattacatataggga

SEQ ID NO: 14 - DNA, 20 bp, Artificial
  Synthetic nucleic acid
attccattgtgaccaggttg

SEQ ID NO: 15 - DNA, 20 bp, Artificial
  Synthetic nucleic acid
ataatgaggcctggaacgga

SEQ ID NO: 16 - DNA, 20 bp, Artificial
  Synthetic nucleic acid
ggttgtccagagctttacgg

SEQ ID NO: 17 - DNA, 20 bp, Artificial
  Synthetic nucleic acid
aagctctgagagcagatagg

SEQ ID NO: 18 - DNA, 20 bp, Artificial
  Synthetic nucleic acid
ttgagttttgctgcagtagg

SEQ ID NO: 19 - DNA, 20 bp, Artificial
  Synthetic nucleic acid
tgatgccctcgattgtcttc

SEQ ID NO: 20 - DNA, 20 bp, Artificial
  Synthetic nucleic acid
actttgagcagctcacaacc

SEQ ID NO: 21 - DNA, 20 bp, Artificial
  Synthetic nucleic acid
ctggcaattgcgatgtacca

SEQ ID NO: 22 - DNA, 20 bp, Artificial
  Synthetic nucleic acid
gtttcaggcccaagagaacc

SEQ ID NO: 23 - DNA, 20 bp, Artificial
  Synthetic nucleic acid
aagagccaggaagaaaccac

SEQ ID NO: 24 - DNA, 20 bp, Artificial
  Synthetic nucleic acid
cttggcaaaactgcaccttc

SEQ ID NO: 25 - DNA, 20 bp, Artificial
  Synthetic nucleic acid
ggagttacaagaggctacca

SEQ ID NO: 26 - DNA, 20 bp, Artificial
  Synthetic nucleic acid
cgagctcacttgaaacgaac

SEQ ID NO: 27 - DNA, 20 bp, Artificial
  Synthetic nucleic acid
tagcaaacacaagcattcgg

SEQ ID NO: 28 - DNA, 20 bp, Artificial
  Synthetic nucleic acid
aggaatcgttaccaaccaca

SEQ ID NO: 29 - DNA, 20 bp, Artificial
  Synthetic nucleic acid
ttccctgacagagaccatag

SEQ ID NO: 30 - DNA, 20 bp, Artificial
  Synthetic nucleic acid
ctgtctgatccatctagcac

SEQ ID NO: 31 - DNA, 20 bp, Artificial
  Synthetic nucleic acid
cctcactattctggtcgtgt

SEQ ID NO: 32 - DNA, 20 bp, Artificial
  Synthetic nucleic acid
gatccgcaagttgattcgag

SEQ ID NO: 33 - DNA, 23 bp, Artificial
  Synthetic nucleic acid
gagaattgcactgcaaccatgag

SEQ ID NO: 34 - DNA, 24 bp, Artificial
  Synthetic nucleic acid
cgattctgaaactcagtccggtaa

SEQ ID NO: 35 - DNA, 20 bp, Artificial
  Synthetic nucleic acid
atccacacagctcagaacag

SEQ ID NO: 36 - DNA, 20 bp, Artificial
  Synthetic nucleic acid
cagcaaatgccaagaccaac

SEQ ID NO: 37 - DNA, 20 bp, Artificial
  Synthetic nucleic acid
cagttaccaggactacgaga

SEQ ID NO: 38 - DNA, 20 bp, Artificial
  Synthetic nucleic acid
gggtgattagctcatgactg

SEQ ID NO: 39 - DNA, 20 bp, Artificial
  Synthetic nucleic acid
cttctatctggttgaacgct

SEQ ID NO: 40 - DNA, 20 bp, Artificial
  Synthetic nucleic acid
cagggaagttgaaatacgca

SEQ ID NO: 41 - DNA, 22 bp, Artificial
  Synthetic nucleic acid
aatcgggcgagagaaaaccgaa

SEQ ID NO: 42 - DNA, 24 bp, Artificial
  Synthetic nucleic acid
gacagttgaaagaggagctcggaa

SEQ ID NO: 43 - DNA, 20 bp, Artificial
  Synthetic nucleic acid
attcaccccaagaacatcca

SEQ ID NO: 44 - DNA, 20 bp, Artificial
  Synthetic nucleic acid
caggattgaggcaagctttc

SEQ ID NO: 45 - DNA, 20 bp, Artificial
  Synthetic nucleic acid
caatgagtacacagcaagac

SEQ ID NO: 46 - DNA, 20 bp, Artificial
  Synthetic nucleic acid
agctctgtgagtaagattcc

SEQ ID NO: 47 - DNA, 24 bp, Artificial
  Synthetic nucleic acid
gacagctccaaacagtttacctca

SEQ ID NO: 48 - DNA, 21 bp, Artificial
  Synthetic nucleic acid
tgtctcgcaggtcatagcgaa

SEQ ID NO: 49 - DNA, 22 bp, Artificial
  Synthetic nucleic acid
gcctggtgagttttatttcgac

SEQ ID NO: 50 - DNA, 21 bp, Artificial
  Synthetic nucleic acid
acgctctcctttcaatcctac

SEQ ID NO: 51 - DNA, 20 bp, Artificial
  Synthetic nucleic acid
ggtttctacggagttaaggg

SEQ ID NO: 52 - DNA, 20 bp, Artificial
  Synthetic nucleic acid
ttcacccttgtactgatctg

SEQ ID NO: 53 - DNA, 20 bp, Artificial
  Synthetic nucleic acid
gagcaaacccgcatgttgga

SEQ ID NO: 54 - DNA, 20 bp, Artificial
  Synthetic nucleic acid
gcaatggtgatggcacggaa

SEQ ID NO: 55 - DNA, 22 bp, Artificial
  Synthetic nucleic acid
gacccagatcatgtttgagacc

SEQ ID NO: 56 - DNA, 20 bp, Artificial
  Synthetic nucleic acid
gtcaccggagtccatcacga

SEQ ID NO: 57 - DNA, 20 bp, Artificial
  Synthetic nucleic acid
accagatcatcattacagga

SEQ ID NO: 58 - DNA, 19 bp, Artificial
  Synthetic nucleic acid
catcattaagtcgagggcc

SEQ ID NO: 59 - DNA, 23 bp, Artificial
  Synthetic nucleic acid
aartacacaacaacaaagtgtgt

SEQ ID NO: 60 - DNA, 24 bp, Artificial
  Synthetic nucleic acid
tccrctccyctyctyctgtgtctg

SEQ ID NO: 61 - RNA, 19 nt, Artificial
  Synthetic nucleic acid
gcacugggcccuguauaua

SEQ ID NO: 62 - RNA, 19 nt, Artificial
  Synthetic nucleic acid
uauggcaagucccgcugua

SEQ ID NO: 63 - RNA, 19 nt, Artificial
  Synthetic nucleic acid
caacagugcagaggugaaa

SEQ ID NO: 64 - RNA, 18 nt, Artificial
  Synthetic nucleic acid
cgaaggagaugguugguc

SEQ ID NO: 65 - RNA, 19 nt, Artificial
  Synthetic nucleic acid
gcaaauguggugagaaaua

SEQ ID NO: 66 - RNA, 19 nt, Artificial
  Synthetic nucleic acid
gaaccgaguguuugacaag

SEQ ID NO: 67 - RNA, 19 nt, Artificial
  Synthetic nucleic acid
ggcagcaccuuuaucuaug

SEQ ID NO: 68 - RNA, 19 nt, Artificial
  Synthetic nucleic acid
acgugaaggaggucaggua

SEQ ID NO: 69 - RNA, 19 nt, Artificial
  Synthetic nucleic acid
gagaagacccucacuagua

SEQ ID NO: 70 - RNA, 19 nt, Artificial
  Synthetic nucleic acid
ccacugaagucuauaugaa

SEQ ID NO: 71 - RNA, 19 nt, Artificial
  Synthetic nucleic acid
gaacaggcccgaguacuaa

SEQ ID NO: 72 - RNA, 19 nt, Artificial
  Synthetic nucleic acid
cgcauaagcuaaagaucua

SEQ ID NO: 73 - RNA, 19 nt, Artificial
  Synthetic nucleic acid
ggaagggucuaaacaguau

SEQ ID NO: 74 - RNA, 19 nt, Artificial
  Synthetic nucleic acid
guaggagcauuacuguuaa

SEQ ID NO: 75 - RNA, 19 nt, Artificial
  Synthetic nucleic acid
gcaacgggauguagauuuc

SEQ ID NO: 76 - RNA, 19 nt, Artificial
  Synthetic nucleic acid
cagaaaaggauuugcgaua

SEQ ID NO: 77 - RNA, 19 nt, Artificial
  Synthetic nucleic acid
gcaauaugcuauggacuau

SEQ ID NO: 78 - RNA, 19 nt, Artificial
  Synthetic nucleic acid
gacuggcaauugcgaugua

SEQ ID NO: 79 - RNA, 19 nt, Artificial
  Synthetic nucleic acid
gagacggaauguuaucaga

SEQ ID NO: 80 - RNA, 19 nt, Artificial
  Synthetic nucleic acid
uagaguguguaaccagauu

SEQ ID NO: 81 - RNA, 19 nt, Artificial
  Synthetic nucleic acid
gagagaauuucccagauug

SEQ ID NO: 82 - RNA, 19 nt, Artificial
  Synthetic nucleic acid
agaaacaccuugaaguaug

SEQ ID NO: 83 - RNA, 19 nt, Artificial
  Synthetic nucleic acid
gaacaacacuggcgaugac

SEQ ID NO: 84 - RNA, 19 nt, Artificial
  Synthetic nucleic acid
gggaagugggagucgaaca

SEQ ID NO: 85 - RNA, 19 nt, Artificial
  Synthetic nucleic acid
gaaagcugcugaacucuuc

SEQ ID NO: 86 - RNA, 19 nt, Artificial
  Synthetic nucleic acid
gaucaucucuggcacaaca

SEQ ID NO: 87 - RNA, 19 nt, Artificial
  Synthetic nucleic acid
gaguaggucugauucauga

SEQ ID NO: 88 - RNA, 19 nt, Artificial
  Synthetic nucleic acid
gaucacaucuggauauuuu

SEQ ID NO: 89 - RNA, 19 nt, Artificial
  Synthetic nucleic acid
gaacaaagcccuggaaguc

SEQ ID NO: 90 - RNA, 19 nt, Artificial
  Synthetic nucleic acid
gggcagugcuacaguauuc

SEQ ID NO: 91 - RNA, 19 nt, Artificial
  Synthetic nucleic acid
gcacuucccaccuaauaaa

SEQ ID NO: 92 - RNA, 19 nt, Artificial
  Synthetic nucleic acid
ugaagccucugauguuuga

SEQ ID NO: 93 - RNA, 19 nt, Artificial
  Synthetic nucleic acid
guuccaagugggacagaua

SEQ ID NO: 94 - RNA, 19 nt, Artificial
  Synthetic nucleic acid
acgauaaaggccagcagua

SEQ ID NO: 95 - RNA, 19 nt, Artificial
  Synthetic nucleic acid
agaauauauugacggaaga

SEQ ID NO: 96 - RNA, 19 nt, Artificial
  Synthetic nucleic acid
aaugggaacuggagagugu

SEQ ID NO: 97 - RNA, 19 nt, Artificial
  Synthetic nucleic acid
cgaagcagcucaaugaaau

SEQ ID NO: 98 - RNA, 19 nt, Artificial
  Synthetic nucleic acid
ggagauagaaguaguccua

SEQ ID NO: 99 - RNA, 19 nt, Artificial
  Synthetic nucleic acid
acacguaccugcagaucua

SEQ ID NO: 100 - RNA, 19 nt, Artificial
  Synthetic nucleic acid
acaggaaggguaccgauua

SEQ ID NO: 101 - RNA, 19 nt, Artificial
  Synthetic nucleic acid
ggucaagcagagagaacga

SEQ ID NO: 102 - RNA, 19 nt, Artificial
  Synthetic nucleic acid
caaccuuguauacgaguca

SEQ ID NO: 103 - RNA, 19 nt, Artificial
  Synthetic nucleic acid
caaccagcauugaccgauu

SEQ ID NO: 104 - RNA, 19 nt, Artificial
  Synthetic nucleic acid
ccugugaccaccuggcgaa

SEQ ID NO: 105 - RNA, 19 nt, Artificial
  Synthetic nucleic acid
ccaaagaauuccagauuaa

SEQ ID NO: 106 - RNA, 19 nt, Artificial
  Synthetic nucleic acid
gcucgaaaguuccuuauga

SEQ ID NO: 107 - RNA, 19 nt, Artificial
  Synthetic nucleic acid
caagugagcucgaaaguuc

SEQ ID NO: 108 - RNA, 19 nt, Artificial
  Synthetic nucleic acid
cgaaaguuccuuaugagau

SEQ ID NO: 109 - RNA, 19 nt, Artificial
  Synthetic nucleic acid
ucacagauguuucgauaaa

SEQ ID NO: 110 - RNA, 19 nt, Artificial
  Synthetic nucleic acid
gaaugggaaucagucauga

SEQ ID NO: 111 - RNA, 19 nt, Artificial
  Synthetic nucleic acid
ccacaaauggaguacaaua

SEQ ID NO: 112 - RNA, 19 nt, Artificial
  Synthetic nucleic acid
cgacauaccggaagacgaa

SEQ ID NO: 113 - RNA, 19 nt, Artificial
  Synthetic nucleic acid
gaagcuauguuuggaauua

SEQ ID NO: 114 - RNA, 19 nt, Artificial
  Synthetic nucleic acid
gaagaggaauguuuaaauc

SEQ ID NO: 115 - RNA, 19 nt, Artificial
  Synthetic nucleic acid
gaucaucgauuuaggauug

SEQ ID NO: 116 - RNA, 19 nt, Artificial
  Synthetic nucleic acid
caacauagccaacauaaau

SEQ ID NO: 117 - RNA, 19 nt, Artificial
  Synthetic nucleic acid
caaauugggugcugcuaua

SEQ ID NO: 118 - RNA, 19 nt, Artificial
  Synthetic nucleic acid
ggagaaagccccaggugua

SEQ ID NO: 119 - RNA, 19 nt, Artificial
  Synthetic nucleic acid
gcaaaagucuuccaaguaa

SEQ ID NO: 120 - RNA, 19 nt, Artificial
  Synthetic nucleic acid
gaacuaauaggacacgcug

SEQ ID NO: 121 - RNA, 19 nt, Artificial
  Synthetic nucleic acid
guaacuggcuuccuuuaaa

SEQ ID NO: 122 - RNA, 19 nt, Artificial
  Synthetic nucleic acid
caugaagucugucgcuaua

SEQ ID NO: 123 - RNA, 19 nt, Artificial
  Synthetic nucleic acid
ggaaauggagcauggaaua

SEQ ID NO: 124 - RNA, 19 nt, Artificial
  Synthetic nucleic acid
gaaacaacgucuggccauu

SEQ ID NO: 125 - RNA, 19 nt, Artificial
  Synthetic nucleic acid
gaagguauuuggucgagua

SEQ ID NO: 126 - RNA, 19 nt, Artificial
  Synthetic nucleic acid
gcacucaccauuaaaucua

SEQ ID NO: 127 - RNA, 19 nt, Artificial
  Synthetic nucleic acid
ggagagagguauaaugauc

SEQ ID NO: 128 - RNA, 19 nt, Artificial
  Synthetic nucleic acid
aaaugucgcuuaaugccua

SEQ ID NO: 129 - RNA, 19 nt, Artificial
  Synthetic nucleic acid
ggacuaugcaaagaaucua

SEQ ID NO: 130 - RNA, 19 nt, Artificial
  Synthetic nucleic acid
gguuuuucaaguacgaaaa

SEQ ID NO: 131 - RNA, 19 nt, Artificial
  Synthetic nucleic acid
gucuaaugagaacuccaga

SEQ ID NO: 132 - RNA, 19 nt, Artificial
  Synthetic nucleic acid
gccuagaacccagucuaau

SEQ ID NO: 133 - RNA, 19 nt, Artificial
  Synthetic nucleic acid
ucacagaucugaagaacau

SEQ ID NO: 134 - RNA, 19 nt, Artificial
  Synthetic nucleic acid
ccaagaaccagagaaaaga

SEQ ID NO: 135 - RNA, 19 nt, Artificial
  Synthetic nucleic acid
uagcgacuaaacacaucaa

SEQ ID NO: 136 - RNA, 19 nt, Artificial
  Synthetic nucleic acid
auguauuggccuguauuag

SEQ ID NO: 137 - RNA, 19 nt, Artificial
  Synthetic nucleic acid
uaaggcuaugaagagauac

SEQ ID NO: 138 - RNA, 19 nt, Artificial
  Synthetic nucleic acid
augaacgugaauugcucaa

(19) United States
(12) Patent Application Publication
(10) Pub. No.: US 2021/0315970 A1
(43) Pub. Date: Oct. 14, 2021

(54) Hepatitis A Virus Replication Inhibitor Targeting mTOR

(71) Applicant: Tokyo Metropolitan Institute of Medical Science, Tokyo (JP)

(72) Inventors: Daisuke Yamane, Tokyo (JP); Asuka Yuuki, Tokyo (JP)

(21) Appl. No.: 17/227,859

(22) Filed: Apr. 12, 2021

(30) Foreign Application Priority Data
Apr. 14, 2020 (JP) 2020072209

(51) Int. Cl. A61K 38/17; A61P 31/14; A61K 31/436; C12Q 1/70

(52) U.S. Cl. CPC A61K 38/1783; A61P 31/14; C12Q 2600/136; C12Q 1/706; A61K 31/436

(57) ABSTRACT

[Problem to be solved] To provide a pharmaceutical composition for treating a disease caused by an RNA virus.

[Solution] A pharmaceutical composition for a disease caused by an RNA virus or an inhibitor of RNA virus replication, comprising retinoic acid receptor responder protein 3 and/or an mTOR inhibitor.

### TECHNICAL FIELD

The present invention relates to a hepatitis A virus replication inhibitor targeting mTOR.

### BACKGROUND ART

Hepatitis A virus (HAV) is a virus with single-stranded RNA genome belonging to the genus Hepatovirus of the family Picornaviridae, and is a pathogen that is transmitted by the fecal-oral route and causes acute hepatitis. Generally, children experience only subclinical or mild symptoms whereas acute hepatitis is caused in adults, which tends to be exacerbated in elderly patients and immunodeficient patients (Non-patent document 1). Hepatitis A is generally cured within 4 to 8 weeks following infection without becoming chronic, with the mortality being low. Hepatitis A occurs mainly in the developing countries, but sporadic outbreaks are also reported globally in the developed countries including the United States, Europe, Japan and Korea. Infection via sexual contact between men is increasing in the urban areas and the number of reported severe cases is increasing as well. Since there is no option for such serious hepatitis A other than symptomatic treatments, there is an urgent need for developing an effective treatment.

### PRIOR ART DOCUMENT

### Non-Patent Document

Non-patent document 1: Jacobsen K H. I Globalization and the Changing Epidemiology of Hepatitis A Virus. Cold Spring Harb Perspect Med. 2018 Oct. 1; 8(10). doi: 10.1101/cshperspect.a031716

### SUMMARY OF INVENTION

### Problem to be Solved by the Invention

The present invention has an objective of providing a substance for inhibiting replication of a hepatitis virus.

In order to solve the above-described problem, the present inventors have gone through intensive studies, and as a result of which succeeded in inhibiting replication of RNA virus HAV, thereby accomplishing the present invention.

### Means for Solving the Problem

Thus, the present invention is as follows.

(1) A pharmaceutical composition for a disease caused by an RNA virus, the pharmaceutical composition comprising retinoic acid receptor responder protein 3 and/or an mTOR inhibitor.

(2) The pharmaceutical composition according to (1), wherein the mTOR inhibitor additionally has an activity of inhibiting phosphatidylinositol 3-kinase.

(3) The pharmaceutical composition according to (1), wherein the mTOR inhibitor is a rapamycin derivative or an mTOR complex inhibitor.

(4) The pharmaceutical composition according to any one of (1)-(3), wherein the disease caused by an RNA virus is hepatitis A, herpangina, hand-foot-and-mouth disease, poliomyelitis or foot-and-mouth disease in swine.

(5) An inhibitor of RNA virus replication, comprising retinoic acid receptor responder protein 3 and/or an mTOR inhibitor.

(6) The replication inhibitor according to (5), wherein the mTOR inhibitor is a dual inhibitor which additionally has an activity of inhibiting phosphatidylinositol 3-kinase.

(7) The replication inhibitor according to (5), wherein the mTOR inhibitor is a rapamycin derivative or an mTOR complex inhibitor.

(8) The replication inhibitor according to any one of (5)-(7), wherein the RNA virus is hepatitis A virus, a coxsackievirus, an enterovirus, a poliovirus or a foot-and-mouth disease virus.

(9) A method for screening an inhibitor of RNA virus replication, the method comprising: bringing a test substance into contact with a cell; and selecting a substance having an activity of inhibiting RNA virus replication by using expression of a gene coding for retinoic acid receptor responder protein 3 in the cell as an indicator.

(10) A method for inhibiting RNA virus replication, the method comprising allowing expression of a gene coding for retinoic acid receptor responder protein 3 in a cell.

(11) The method according to either one of (9) and (10), wherein the RNA virus is hepatitis A virus, a coxsackievirus, an enterovirus, a poliovirus or a foot-and-mouth disease virus.

### Effect of the Invention

The pharmaceutical composition and the inhibitor of the present invention are capable of inhibiting replication of an RNA virus, in particular, hepatitis A virus, and thus can be used as a therapeutic agent for hepatitis A.

### BRIEF DESCRIPTION OF THE DRAWINGS

FIG. 1a Diagrams showing that IRF1 restricts RNA virus infection in hepatocytes (FIGS. 1a-1j). Intracellular HAV RNA 5 days post-inoculation in PH5CH8 cells transduced with lentiviruses expressing shRNAs targeting different genes. *P<0.05, **P<0.01 vs. control group (two-way analysis of variance with Dunnett's multiple comparison test).

FIG. 1b Diagrams showing kinetics of HAV RNA replication over 5 days in PH5CH8 cells expressing sgRNAs targeting IRF1 vs IRF3 vs RELA. *P<0.05, **P<0.01 vs. control (two-way analysis of variance with Dunnett's multiple comparison test). Shown on the left are immunoblots of IRF1, IRF3, IRF7 and RelA in knockout cells. Shown on the right are viral titers 5 days post-inoculation. **P<0.01 compared to the control group (one-way analysis of variance with Dunnett's multiple comparison test). GAPDH: glyceraldehyde-3-phosphate dehydrogenase.

FIG. 1c Diagrams showing fecal shedding of HAV on Day 5 and Day 7 and HAV RNA abundance in livers on Day 3 and Day 7 following inoculation of the virus in wild-type vs Irf1^{-/-} C57BL/6 mice. Left: data were pooled from two different time points. Right: each mark represents single animal. *P<0.05 compared to wild type (two-sided unpaired Mann-Whitney's U test). GE: number of genome equivalents.

FIG. 1d Diagrams showing HAV RNA abundance 5 days post-inoculation in PH5CH8 cells expressing IFNAR1 or IFNLR1 sgRNAs vs IRF1 sgRNA. Shown on the left are immunoblots of IFNAR1 and ISG (MDA5 and OAS1) induced by either recombinant IFN-α (24 hours 100 U ml⁻¹) or IFN-kl (10 ng ml-t) in these IFN receptor-knockout cells. **P<0.01 compared to the control group (one-way analysis of variance with Dunnett's multiple comparison test). ISG: interferon-stimulated genes.

FIG. 1e HAV RNA abundance 5 days post-inoculation in PH5CH8 cells expressing STAT1 sgRNA or both STAT1 and IRF1 sgRNAs (right). **P<0.01 compared to the control group (one-way analysis of variance with Dunnett's multiple comparison test). Immunoblots showing that there is no ISG expression in response to type I and type III IFNs (left).

FIG. 1f HAV RNA 5 days post-inoculation in PH5CH8 cells in the continued presence of Jak inhibitors, i.e., 3 μM ruxolitinib or 0.3 μM pyridone 6 (left). *P<0.05, **P<0.01 vs. control (one-way analysis of variance with Dunnett's multiple comparison test or two-sided Student's t test). After IRF1 was knocked out, HAV replication enhanced in the presence of ruxolitinib (right). *P<0.05, **P<0.01 vs. control (two-sided unpaired Student's t test).

FIG. 1g Diagrams showing the influence of IRF1 double-knockout in the absence of MAVS or IRF3 on HAV replication. Relative HAV RNA abundance 5 days post-inoculation, provided that HAV RNA abundance in the absence of IRF1 sgRNA was set to 1 (right). Immunoblots are shown on left. **P<0.01 compared to the control group (two-sided unpaired Student's t test).

FIG. 1h Immunoblots of IRF1 in control and IRF1-knockout Huh-7.5 cells (left). GLuc secreted from the Huh-7.5 cells infected with JFH1-QL/GLuc virus (103 FFU ml⁻¹) over the following 96 hours (right). **P<0.01 compared to the control group (two-way analysis of variance with Dunnett's multiple comparison test).

FIG. 1i HAV RNA abundance over 48 hours in IRF1 sgRNA-expressing cells vs control Huh-7.5 cells infected at MOI=1. **P<0.01 compared to the control group (two-sided unpaired Student's t test). MOI: multiplicity of infection.

FIG. 1j DENV and ZIKV RNA abundance over 48 hours in IRF1 siRNA- vs control siRNA-transfected Huh-7.5 cells which were infected at MOI=1. *P<0.05, **P<0.01 vs. control (two-sided unpaired Student's t test). Data present mean±s.d. from three independent experiments (a, b, d-h, j), or as mean±s.d. from three technical replicates representative of two independent experiments (c, i).

FIG. 2a Diagrams showing that IRF1 constitutively activates baseline levels of transcriptions of PRDIII-I- and ISRE-dependent antiviral genes (FIGS. 2a-2f). Dual-luciferase reporter analysis for 4×PRDIII-I-Luc (upper panels) and ISRE-Luc (lower panels) activities in mock- (left panels) and HAV-infected (right panels) PH5CH8 cells. Promoter activities in IRF1-sgRNA (#1 and 2)-expressing cells were significantly different from those in control or IRF3-sgRNA-expressing cells (P<0.01, two-way analysis of variance with Dunnett's multiple comparison test).

FIG. 2b Dose-response analysis of PRDIII-I (upper) and ISRE (lower) activities in wild-type PH5CH8 cells infected with HAV and SeV. *p<0.05, **p<0.01 vs mock (one-way analysis of variance with Dunnett's multiple comparisons test). SeV: Sendai virus.

FIG. 2c Dual-luciferase reporter analysis of 4×PRDIII-I-Luc (upper) and ISRE-Luc (lower) activities in mock-infected Huh-7.5 cells. SeV does not activate these promoters in Huh-7.5 cells. **P<0.01 compared to the control group (two-way analysis of variance with Dunnett's multiple comparison test).

FIG. 2d Nuclear localization of IRF1 in two different hepatocyte cell lines and primary human fetal hepatocytes. Data are representative of two independent experiments. Scale bar: 20 μm.

FIG. 2e HAV RNA 24 hours post-inoculation in Huh-7.5 cells expressing IRF1 sgRNA that were pretreated with actinomycin D (5 μg ml⁻¹) for 30 minutes. *P<0.05, **P<0.01 vs. control (two-sided unpaired Student's t test).

FIG. 2f DENV RNA abundance 18 hours post-inoculation or ZIKV RNA abundance 24 hours post-inoculation in IRF1-knockdown Huh-7.5 cells that were pretreated with actinomycin D (5 μg ml-1) for 30 minutes. *P<0.05, **P<0.01 vs. control (two-sided unpaired Student's t test). Data present mean±s.d. from three technical replicates representative of two independent experiments (a-d) or from two independent experiments (e,f).

FIG. 3a Diagrams showing antiviral activities of IRF1 effector genes identified by high-throughput RNA-seq against different viruses (FIGS. 3a-3l). Venn diagrams showing numbers of genes with expression change of 2-fold in each of sgRNA-expressing cells.

FIG. 3b List of genes that were reduced by 2-fold or more in IRF1 sgRNA-expressing cells compared to IRF3 sgRNA-expressing cells. Indicated values are means of fold changes of the genes expressed in cells transduced with two independent IRF1 sgRNA (left) or IRF3 sgRNA (right).

FIG. 3c Validation of RNA-seq results by RT-qPCR assays of RNA extracted from uninfected vs. HAV-infected PH5CH8 cells. The scatter plots show ratios of indicated gene transcripts expressed in IRF1 sgRNA- vs. control sgRNA-expressing PH5CH8 cells in HAV-infected cells (y-axis) and mock-infected cells (x-axis).

FIG. 3d Heat map showing relative abundance of indicated genes in uninfected PH5CH8 cells expressing sgRNA targeting IRF1 or IRF3 as determined by RT-qPCR assays.

FIG. 3e Relative HAV RNA abundance 5 days post-infection in PH5CH8 cells transfected with siRNA targeting different IRF1 effector genes. **P<0.01 vs. control.

FIG. 3f Independent validation of the siRNA results and the combination of four siRNAs. *P<0.05, **P<0.01 vs. control.

FIG. 3g Relative GLuc activity 3 days post-inoculation in HCV-infected Huh-7.5 cells. *P<0.05, **P<0.01 vs. control.

FIG. 3h Independent validation of the siRNA results and the combination of three siRNAs. *P<0.05, **P<0.01 vs. control.

FIG. 3i Relative DENV RNA levels 24 hours post-inoculation in infected Huh-7.5 cells. P<0.05 vs. control.

FIG. 3j Independent validation of the siRNA results and combination of two siRNAs. *P<0.05, **P<0.01 vs. control.

FIG. 3k ZIKV RNA levels 24 hours post-inoculation in infected Huh-7.5 cells. *P<0.05, **P<0.01 vs. control.

FIG. 3l Independent validation of the siRNA results and combination of two siRNAs. **P<0.01 vs. control. Data present mean±s.d. from three independent experiments (e-g, i-l), or mean±s.d. from three technical replicates representative of two independent experiments (h). P values were derived using one-way analysis of variance with Dunnett's multiple comparison test (e, g, i-l) or two-sided unpaired Student's t test (f, h).

FIG. 4a Diagrams showing that RARRES3 acyltransferase whose transcription is regulated by IRF1 restricts HAV replication by down-regulating mTOR (FIGS. 4a-4i). Lentivirus transduction of active RARRES3 restricts HAV infection in PH5CH8 cells expressing IRF1 sgRNA no. 2 (left panel) or Huh-7.5 cells (right panel). While RARRES3 inhibited HAV infection in both cell lines, catalytically-inactive RARRES3/C113S mutant did not inhibit HAV infection. *p<0.05, **p<0.01 vs. vector control (two-way analysis of variance with Dunnett's multiple comparison test).

FIG. 4b Huh-7.5 cells stably expressing indicated lentiviral vectors were infected with HAV expressing NLuc, and treated with 30 μM 2′CMA (direct acting antiviral (DAA)) or dimethyl sulfoxide (DMSO) as a vehicle control. NLuc activities at indicated time points following infection are shown. **P<0.01 (two-way analysis of variance with Dunnett's multiple comparison test).

FIG. 4c Transition of FLuc activities following transfection of subgenomic HAV-Luc RNA or its replication incompetent mutant (A3D) RNA in Huh-7.5 cells expressing wild-type RARRES3 or inactive RARRES3/C113S mutant. **P<0.01 vs. vector control group (two-way analysis of variance with Dunnett's multiple comparison test).

FIG. 4d Infection of HAV/NLuc in Huh-7.5 cells expressing PLAAT4/RARRES3 sgRNA.

Immunoblots are shown on top. **P<0.01 vs. vector control group (two-way analysis of variance with Dunnett's multiple comparison test).

FIG. 4e Steady-state levels of mTOR-related factors in Huh-7.5 cells stably expressing RARRES3 and RARRES3/C113S.

FIG. 4f Immunoblots of Huh-7.5/RARRES3 cells transfected with P70-S6K siRNA.

FIG. 4g Phosphorylation of p70-S6K and mTOR in Huh-7.5 cells expressing IRF1 sgRNA.

FIG. 4h Influence of mTOR inhibitors on HAV/NLuc replication and cell viability.

FIG. 4i Inhibition of subgenomic HAV/NLuc RNA replication in the transfected Huh-7.5 cells by three different mTOR inhibitors and DAA (30 μM 2′CMA). **P<0.01 vs. DMSO control group (one-way analysis of variance with Dunnett's multiple comparison test).

FIG. 5 Diagrams showing suppression of HAV replication by mTOR/PI3K dual inhibitors.

FIG. 6 Diagram showing results from validation of an antiviral effect of Pictilisib in an infected mouse model.

### MODES FOR CARRYING OUT THE INVENTION

The present invention relates to a pharmaceutical composition for a disease caused by an RNA virus and to an inhibitor of RNA virus replication, each comprising retinoic acid receptor responder protein 3 and/or an mTOR inhibitor.

1. Overview

(1) Summary

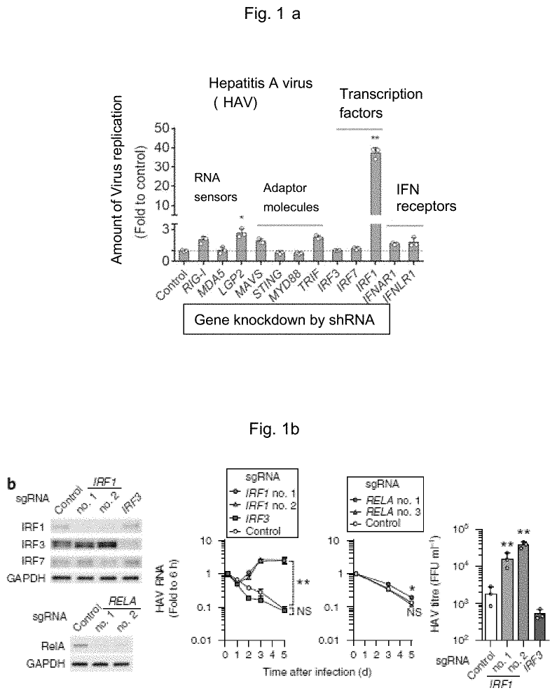

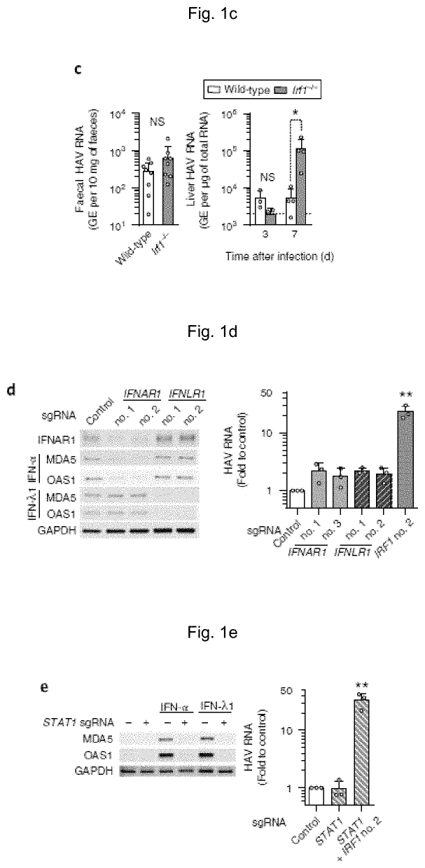

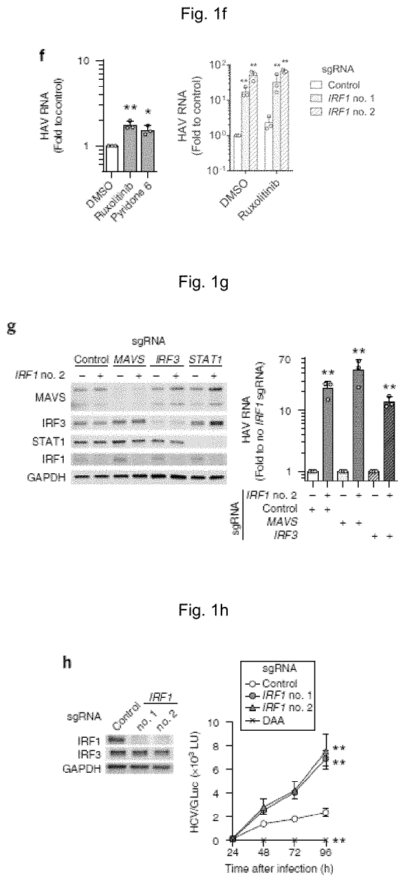

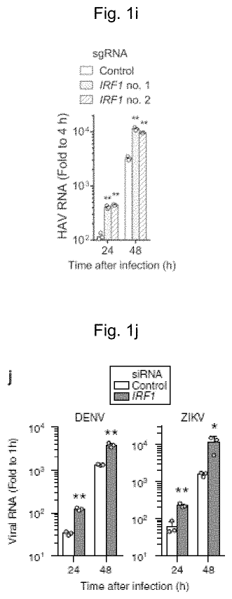

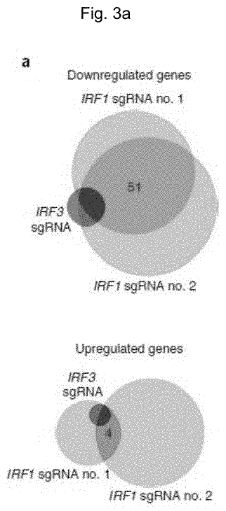

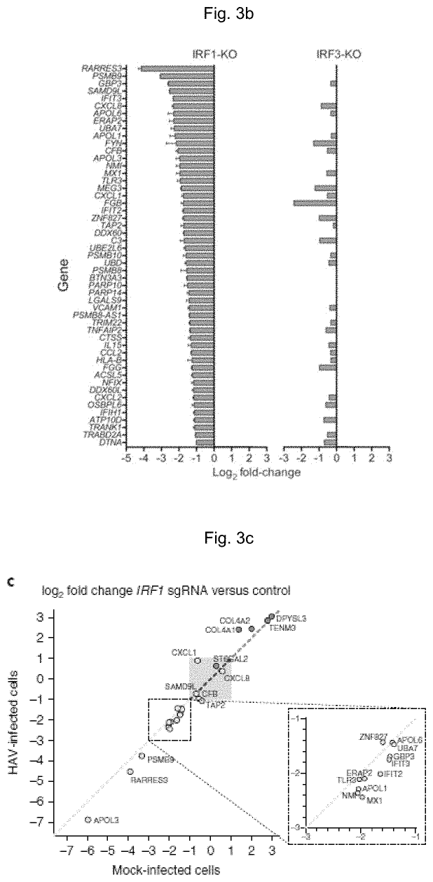

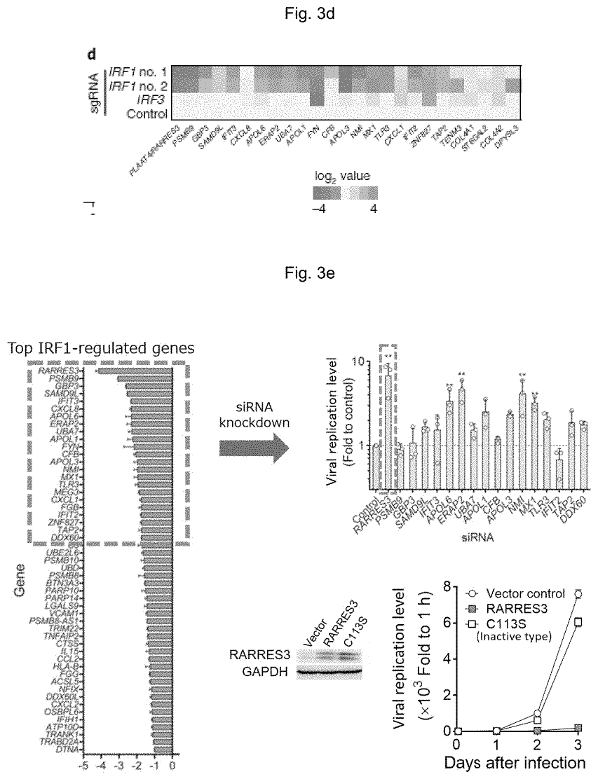

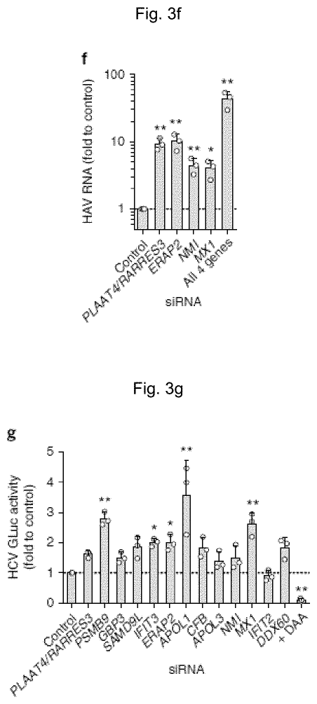

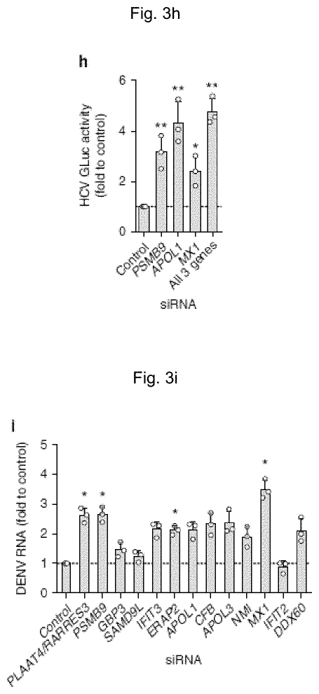

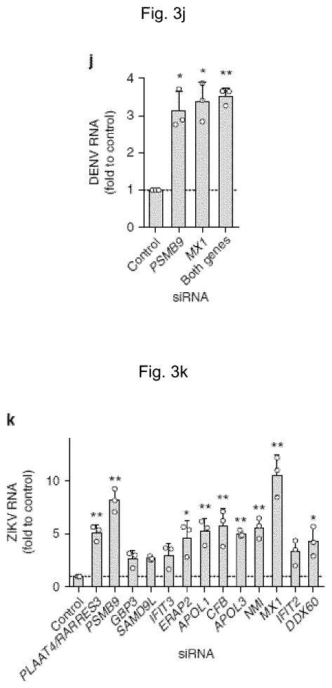

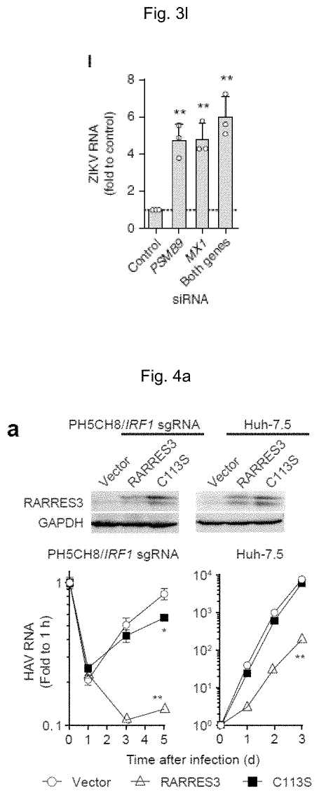

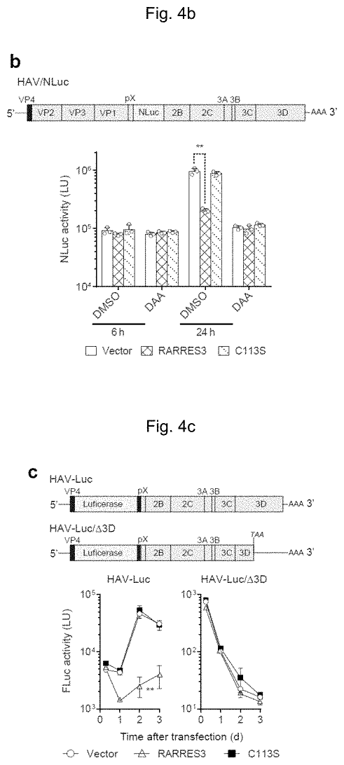

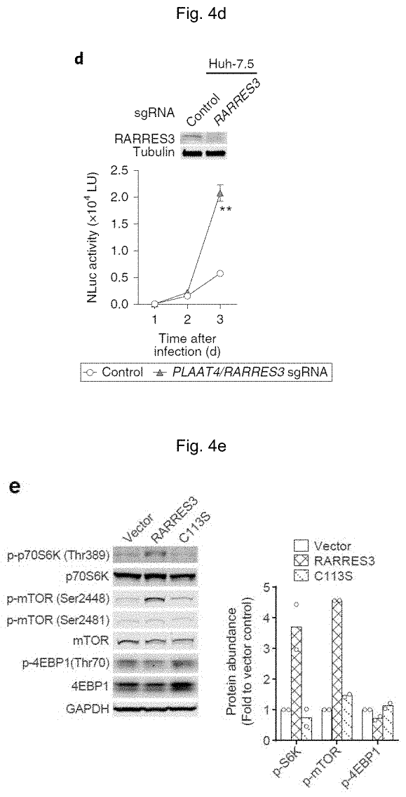

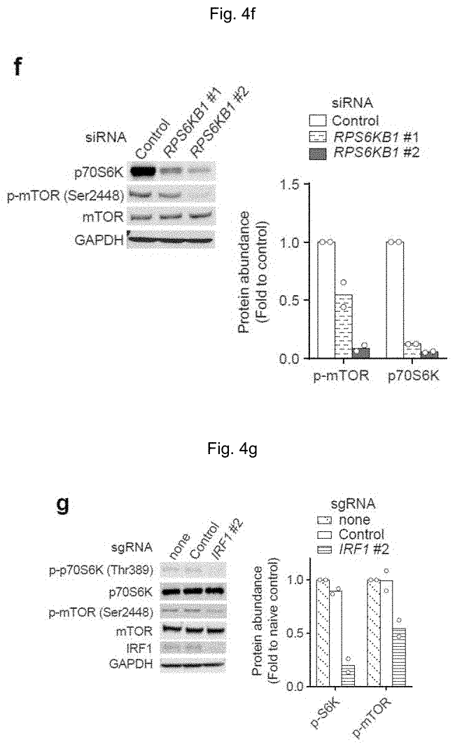

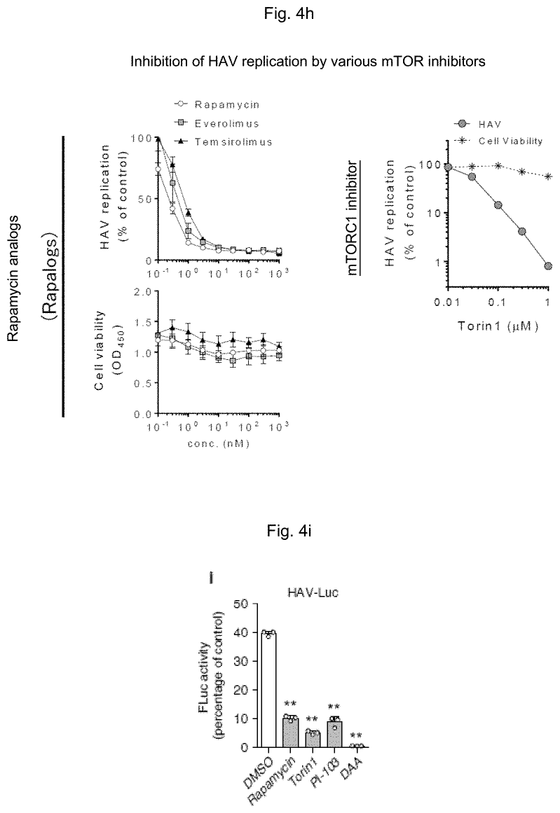

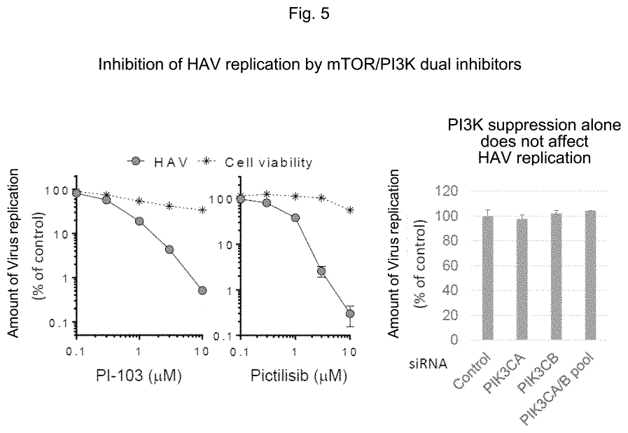

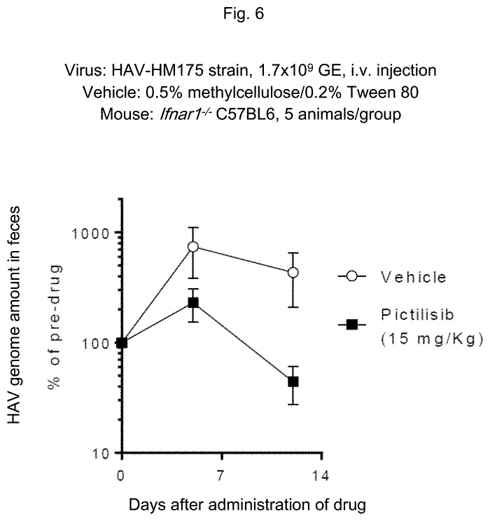

Hepatitis A caused by hepatitis A virus (HAV) infection not only occurs frequently in the developing countries but sporadic outbreaks of hepatitis A are also seen in the developed countries, where an increase in the number of severe cases has been a problem (Reference 1). Nevertheless, there is no choice in hepatitis A treatment other than symptomatic treatments and thus development of an effective antiviral therapy is urgent. Signaling pathways that suppress hepatitis virus replication were analyzed using immortalized primary hepatocytes retaining innate immune signals. As a result, interferon regulatory factor 1 (IRF1) was found to strongly suppress HAV replication (FIG. 1a), and identified 51 IRF1 target genes by RNA-seq analysis (FIGS. 3a and 3b). Among others, RARRES3 that was most strongly induced by IRF1 was found to suppress HAV genome replication via suppression of mTOR activity through its phospholipase A activity (FIGS. 3e, 4b, 4c and 4e). In addition, mTOR inhibitors including rapamycin, rapalogs and Torin-1 were found to be competent drug candidates that mimic the antiviral function of RARRES3 (FIG. 4h). While inhibition of PI3K alone did not suppress HAV at all, use of Pictilisib or PI-103, dual inhibitors that are capable of suppressing both mTOR and PI3K additionally suppressed virus replication to one-tenth or less compared to rapalogs that target mTOR alone (FIG. 5). Furthermore, virus replication was found to be suppressed strongly by Torin-1, an inhibitor of functional complex formed by mTOR (FIG. 4h). Since these drugs efficiently suppressed virus replication at concentrations that did not affect cell viability, and since Pictilisib exhibited the antiviral effect in an infected mouse model (FIG. 6), they are considered to be effective therapeutic agents for hepatitis A.

(2) Methods

(2-1) Analysis of Signaling Pathway that Suppresses Hepatitis Virus Replication

In order to elucidate antiviral signaling pathway in liver, immortalized hepatocytes (PH5CH8) known to express normal antiviral signal factors were used to prepare cells in which a set of antiviral signal genes up from an RNA sensor protein that recognize the RNA virus genomes down to transcription factors and interferon receptors that were activated downstream (RIG-I, MDA5, LGP2, MAVS, TRIF, STING, MYD88, IRF1, IRF3, IRF7, IFNAR1, IFNLR1) were stably knocked down using shRNAs. These knockdown cells were infected with HAV (18f strain) and virus replication levels after infection were analyzed using real-time PCR to identify the antiviral signaling pathways (Reference 2).

(2-2) Preparation of IRF1-Knockout Cells and Identification of IRF1 Target Genes

In order to elucidate the mechanism of action of IRF1 that was found to strongly suppress HAV, CRISPR/Cas9 was used to knock out IRF1 expression, and the host mRNA expression profile was compared to control cells using RNA-seq analysis, thereby identifying genes with specifically and significantly reduced mRNA expression levels in the IRF1-knockout cells as IRF1 target genes.

(2-3) Analysis of Antiviral Functions of IRF1-Regulated Genes

For top twenty or so IRF1-regulated genes, siRNAs were transfected into PH5CH8 cells using Lipofectamine RNAiMAX (Thermo Scientific) to knock down their expression to see their influence on virus replication. The cells were infected with virus on the day after the siRNA transfection and the viral RNA abundance were determined at 4 days post infection using real-time PCR. Moreover, for the IRF1-regulated gene RARRES3 and a phospholipase-A inactive C113S mutant, lentiviral vectors were introduced into hepatoma-derived cells (HuH-7) to prepare stably expressing cells by hygromycin (300 μg/ml) selection.

(2-4) Analysis of Signaling Pathway of RARRES3 that Suppresses HAV

HuH-7 cell lysates expressing wild-type RARRES3 and inactive C113S mutant were harvested to carry out signal analysis by Western blotting using antibodies that can detect mTOR signal-related proteins (Cell Signaling Technologies). In addition, the replication levels after viral infection were determined in detail using HAV expressing NanoLuc reporter (18f strain, HAV/NLuc) to analyze mechanism of action in the viral life cycle (entry, genomic translation and replication).

(2-5) Analysis of Anti-HAV Effect of mTOR Inhibitors

HuH-7 cells were infected with HAV/NLuc and drugs were given 1 hour after infection. 48 hours later, NanoLuc activities were measured to evaluate the virus replication levels. In addition, WST-8 reagent (Cell Counting Kit-8, Dojindo) was used to evaluate cell viability after the drug treatment.

(2-6) Analysis Using HAV-Infected Mouse Models

Type-I interferon receptor (Ifnar1)-knockout C57BL/6 mice were infected by tail vein injection of the virus equivalent to 1.7×10⁹ genome copy number. Pictilisib (15 mg/kg) was administered 5 days after infection and viral RNA contained in the later shed feces was quantified by real-time PCR to analyze the antiviral action of the drug (Reference 3).

(3) Results

(3-1) Using immortalized hepatocyte PH5CH8 cells that retain the antiviral-response capacity of hepatocytes, known antiviral signal genes were knocked down using shRNAs. As a result, the host factors and the interferon receptors that mediate the function of the RNA sensor proteins increased virus replication about 2-3-fold whereas the virus replication level was increased 30-fold or higher in cells in which a transcription factor IRF1 was knocked down, revealing that IRF1 had the strongest anti-HAV action among these genes (Figure Ta).

(3-2) mRNA expression profiles of two different IRF1-knockout cells and control cells introduced with a control vector alone were analyzed two days after the HAV infection by RNA-seq analysis. As a result, fifty-one IRF1-regulated genes down-regulated by IRF1 knockout were identified (FIG. 3a).

(3-3) Top twenty or so genes among the IRF1-regulated genes were knocked down in cells using siRNA, and these cells were infected with HAV to assay viral RNA replication levels by real-time PCR. As a result, multiple genes showed significant increase in the virus replication levels but the largest increase in the replication was observed in cells in which RARRES3, a protein possessing phospholipase A activity, was knocked down (FIG. 3e). Thus, RARRES3 was found to be one of the genes that mediate antiviral function of IRF1. Furthermore, since HAV replication was strongly suppressed in cells overexpressing RARRES3 while no anti-HAV effect was observed in cells expressing phospholipase A activity-defective C113S mutant, phospholipase A activity was found to be indispensable for the antiviral function of RARRES3.

(3-4) Protein expression levels of mTOR-related factors in RARRES3- and inactive mutant C113S-expressing cells were analyzed using specific antibodies. RARRES3 was found to activate p70S6K in its phospholipase A activity-dependent manner and suppresses mTOR activity via phosphorylation of mTOR Ser2448. Furthermore, experiments using NanoLuc reporter virus and subgenomic replicon RNA revealed that RARRES3 suppressed genome replication after the viral entry (FIGS. 4b, 4c and 4e).

(3-5) Rapalogs as mTOR inhibitors (rapamycin, everolimus, temsirolimus) and Torin-1 as an inhibitor of mTOR complex, were added to HuH-7 cells that were infected with HAV expressing NanoLuc to determine NanoLuc activities at 48 hours post-infection. As a result, suppression of virus replication was observed in a concentration-dependent manner without having an influence on the cell viability, where the rapamycin analogs reduced the viral replication level to one-twentieth and Torin-1 reduced it to one-hundredth (FIG. 4h). Dual inhibitors Pictilisib and PI-103 that simultaneously suppress mTOR and PI3K also showed strong anti-HAV activity and reduced the virus replication level to about one-thousandth at the highest dose (10 μM) (FIG. 5). Since no change in virus replication was observed by suppression of PI3K alone, it was considered that dual inhibitors elicit stronger suppression of the mTOR function via suppressing PI3K-mediated reactivation of mTOR.

(3-6) Pictilisib that exhibited the strongest virus replication suppression among the mTOR inhibitors was used to validate the antiviral effect in an infected mouse model. The drug was orally administered from Day 5 following the infection and continuously administered daily for 14-consecutive days except Day 9 and Day 16. The viral RNA levels shed in the feces reflect the viral load in the liver and thus the viral RNA levels in the feces were quantified. As a result, the viral RNA level was reduced to about one-tenth by administration of the drug, confirming its antiviral effect (FIG. 6).

(4) Discussion

Although there has been no established therapeutic method for hepatitis A, HAV replication was shown to be effectively suppressed at a concentration with no noticeable cytotoxicity by targeting mTOR. In addition to a rapalog, everolimus, which is commercially available as an anticancer agent from Novartis under the name of “Afinitor”, Pictilisib has also undergone a phase II trial and is confirmed to be safe to human. Until now, mTOR inhibitors have been generally recognized as immunosuppressants and their use against viral infection has been prohibited because, for example, reactivation of hepatitis B virus and enhanced replication of hepatitis C and E viruses are reported as a consequence of their immunosuppressive effects and autophagy induction (Reference 4). Since, however, potent suppressive effects on HAV replication were observed in cultured cells and an infected animal model, they could be highly effective options for treating specific viral infections. Since mTOR inhibitors are also reported to have suppressive effects on human herpesvirus-8, cytomegalovirus, polyomavirus and rotavirus and since autophagy induced by mTOR inhibitors is reported to suppress human immunodeficiency virus (Reference 5), they could be therapeutic agents effective against pathogens other than HAV.

### (5) References

(1) Jacobsen K H. I Globalization and the Changing Epidemiology of Hepatitis A Virus. Cold Spring Harb Perspect Med. 2018 Oct. 1; 8(10). doi: 10.1101/cshperspect.a031716.
(2) Yamane D, Feng H, Rivera-Serrano E E, Selitsky S R, Hirai-Yuki A, Das A, McKnight K L, Misumi I, Hensley L, Lovell W, Gonzalez-Lopez O, Suzuki R, Matsuda M, Nakanishi H, Ohto-Nakanishi T, Hishiki T, Wauthier E, Oikawa T, Morita K, Reid L M, Sethupathy P, Kohara M, Whitmire J K, Lemon S M. Basal expression of interferon regulatory factor 1 drives intrinsic hepatocyte resistance to multiple RNA viruses. Nat Microbiol. 2019 July; 4(7):1096-1104. doi: 10.1038/s41564-019-0425-6.
(3) Hirai-Yuki A, Hensley L, McGivem D R, Gonzalez-Lopez O, Das A, Feng H, Sun L, Wilson J E, Hu F, Feng Z, Lovell W, Misumi I, Ting J P, Montgomery S, Cullen J, Whitmire J K, Lemon S M. MAVS-dependent host species range and pathogenicity of human hepatitis A virus. Science. 2016 Sep. 30; 353 (6307): 1541-1545.
(4) Sema Sezgin Goksu, Serife Bilal, and Hasan Senol Coskun. Hepatitis B reactivation related to everolimus. 2013 Jan. 27; 5(1): 43-45. doi: 10.4254/wjh.v5.i1.43.
(5) Sagnier S, Daussy C F, Borel S, Robert-Hebmann V, Faure M, Blanchet F P, Beaumelle B, Biard-Piechaczyk M, Espert L. Autophagy restricts HIV-1 infection by selectively degrading Tat in CD4+ T lymphocytes. J Virol. 2015 January; 89(1): 615-25. doi: 10.1128/JVI.02174-14.

2. Pharmaceutical Composition and Replication Inhibitor

Active elements of a pharmaceutical composition and a replication inhibitor of the present invention are retinoic acid receptor responder protein 3 (RARRES3) and/or an mTOR inhibitor.

RARRES3 refers to retinoic acid receptor responder protein 3 whose amino acid sequence and nucleotide sequence are registered in the database (Accession number: NM_004585).

According to the present invention, RARRES3 may be a protein having phospholipase A activity which comprises the amino acid sequence represented by SEQ ID NO:2 or said amino acid sequence with deletion, substitution or addition of one or several amino acids. A protein having such an amino acid sequence can be obtained by a common gene engineering technique. For example, DNA coding for RARRES3 (SEQ ID NO:1) is designed and synthesized. This design and synthesis can be carried out, for example, by a PCR technique using a vector or the like containing full-length RARRES3 gene as a template and primers that are designed to synthesize a desired DNA region. Then, the above-described DNA is linked to an appropriate vector to obtain a recombinant vector for protein expression, which, in turn, is introduced into a host such that the gene of interest is expressed to obtain a transformant. Subsequently, the transformant is cultured to obtain RARRES3 from the culture (Sambrook J. et al., Molecular Cloning, A Laboratory Manual (4th edition) (Cold Spring Harbor Laboratory Press (2012)).

mTOR (mammalian target of rapamycin) is serine/threonine kinase identified as a target molecule of a macrolide-based antibiotic rapamycin, and serves as a regulatory factor in cell division, growth and survival. Inhibitors of mTOR are currently known as immunosuppressants and antitumor drugs.

According to the present invention, mTOR inhibitors can be used for diseases caused by RNA viruses. Examples of mTOR inhibitors include rapamycin derivatives and mTOR complex inhibitors.

Examples of rapamycin derivatives include, but not limited to, sirolimus, everolimus and temsirolimus.

Moreover, examples of mTOR complex inhibitors include, but not limited to, Torin-1 (1-[4-[4-(1-oxopropyl)-1-piperazinyl]-3-(trifluoromethyl)phenyl]-9-(3-quinolinyl)-benzo[h]-1,6-naphthyridin-2(1H)-one), Sapanisertib and AZD 8055.

Furthermore, according to the present invention, dual inhibitors that can simultaneously suppress mTOR and phosphatidylinositol 3-kinase (PI3K) can be used. Examples of such dual inhibitors include Pictilisib, PI-103, Dactolisib, BGT226, SF1126, PKI-587, PF-04691502, Panulisib and XL765.

These rapamycin derivatives, mTOR complex inhibitors and dual inhibitors are available from Selleck, Chemscene, Sigma-Aldrich, Tocris and else.

The pharmaceutical composition of the present invention may use either RARRES3 or mTOR inhibitor alone or may use both of them in combination. The phrase “use in combination” means both of them are used in the same course of therapeutic protocol, and does not necessarily mean that they are used at the same time. Accordingly, they may be administered, for example, in a schedule where administration of RARRES3 is followed by administration of an mTOR inhibitor after a predetermined period of time (for example, two days).

The pharmaceutical composition of the present invention may be either in oral dosage form or parenteral dosage form. These dosage forms may be formulated by a common technique and may contain pharmaceutically acceptable carriers and additives.

Examples of such carriers and additives include water, acetic acid, pharmaceutically acceptable organic solvents, collagen, polyvinyl alcohol, polyvinylpyrrolidone, carboxy vinyl polymers, carboxymethyl cellulose sodium, sodium polyacrylate, sodium alginate, water-soluble dextran, sodium carboxymethyl starch, pectin, methyl cellulose, ethyl cellulose, xanthan gum, gum arabic, casein, agar, polyethylene glycol, diglycerin, glycerin, propylene glycol, petroleumjelly, paraffin, stearyl alcohol, stearic acid, human serum albumin, mannitol, sorbitol, lactose, surfactants accepted as pharmaceutical additives and else.

The above-mentioned additives are selected alone or in a suitable combination according to the dosage form of the pharmaceutical composition of the present invention. The dosage form may be tablets, capsules, fine granules, powder, granules, liquid, syrup or the like for oral administration or a suitable dosage form.

Examples of the dosage form for parenteral administration include injectable agents, aerosols, topical medications and externally applied agents. In the case of injectable dosage forms, they may be systemically or locally administered, for example, by intravenous injection such as a drip, subcutaneous injection, intraperitoneal injection or the like.

If the composition is to be used, for example, as an injectable formulation, the pharmaceutical composition of the present invention is dissolved in a solvent (for example, physiological saline, buffer, glucose solution, etc.), which is then added with a suitable additive (human serum albumin, polyethylene glycol, cyclodextrin conjugate, etc.). Alternatively, the composition may be lyophilized to give a dosage form that can be dissolved upon use.

Examples of excipient for lyophilization include sugar alcohols such as mannitol or glucose and sugars.

The dose of the pharmaceutical composition or the inhibitor of the present invention varies depending on age, sex, symptom, administration route, number of dose and dosage form. For example, the daily dose may be 1 mg-120,000 mg, preferably 2.5 mg-10 mg for an adult (60 kg). The administration method is suitably selected according to age and symptoms of the patient.

The dose may be given, for example, once a day or 2-3 times a day at a dosing interval of several days.

The pharmaceutical composition and the inhibitor of the present invention can be used as an antiviral agent, in particular, an antiviral agent against an RNA virus.

RNA virus is a virus that has genome consisting of ribonucleic acids (RNA), where there are kinds of viruses which express genetic information from genomic RNA without being mediated by DNA, and kinds of viruses which cause genomic RNA to make copies of DNA by means of reverse transcriptase so the genetic information, in turn, is read out from the DNA. The latter is particularly referred to as retroviruses.

RNA viruses may further be classified into double-stranded RNA viruses (dsRNAs), positive-sense single stranded RNA viruses (+strand type) and negative-sense single-stranded RNA viruses (−strand type).

According to the present invention, the kinds of the targeted diseases may be diseases caused by RNA viruses (for example, viruses belonging to the family Picornaviridae), such as hepatitis A, herpangina, hand-foot-and-mouth disease, poliomyelitis and foot-and-mouth disease in swine.

3. Screening Method and Method for Inhibiting Replication

As described above, RARRES3 was found to be one of the genes that mediate antiviral function of IRF1. In addition, since replication of HAV was strongly suppressed in cells overexpressing RARRES3 whereas no anti-HAV effect was observed in cells expressing phospholipase A activity-defective C113S mutant, phospholipase A activity was found to be indispensable to antiviral function of RARRES3.

Therefore, according to the present invention, expression of RARRES3 was used as an indicator to screen for a substance having an activity of inhibiting RNA virus replication. The present invention also provides a method for inhibiting RNA virus replication, the method comprising allowing expression of a gene coding for retinoic acid receptor responder protein 3 in the cells.

The screening method of the present invention comprises the steps of: bringing a test substance into contact with cells having RARRES3 gene or a biomaterial collected from an animal having RARRES3 gene (for example, non-human animal-derived cells, Vero cells, etc.); then, determining the expression level of RARRES3 gene; and, utilizing the obtained determination as an indicator, selecting a substance that inhibits RNA virus replication. In this selection step, the test substance may be actually applied to an RNA virus to validate the level of RNA virus replication.

According to the present invention, if the expression level of RARRES3 gene after bringing the test substance into contact therewith is higher than the expression level of RARRES3 gene without bringing the test substance into contact therewith (control), this substance is selected as a substance that inhibits RNA virus replication. Here, the method for confirming the expression level is not particularly limited. For example, hybridization using a probe for RARRES3 gene, an immunoblotting assay using an antibody against RARRES3 or the like can be employed.

According to the present invention, test substances as candidates of screening (candidate substances) are not particularly limited and examples thereof include peptides, proteins, DNAs, non-peptide compounds, synthetic compounds, fermentation products, cell extracts, plant extracts and the like, where such compounds may be novel compounds or known compounds. These test substances may be in salt forms, in which case, salts of the test substances may be salts formed with a physiologically acceptable acid (for example, an inorganic acid) or base (for example, an organic acid). Examples of such salts include salts formed with inorganic acids (for example, hydrochloric acid, phosphoric acid, hydrobromic acid or sulfuric acid), or salts formed with organic acids (for example, acetic acid, formic acid, propionic acid, fumaric acid, maleic acid, succinic acid, tartaric acid, citric acid, malic acid, oxalic acid, benzoic acid, methanesulfonic acid or benzenesulfonic acid). The test substance may be a single substance or a mixture (including a library or the like). Examples of a library including multiple test substances include a synthetic compound library (combinatorial library, etc.) and a peptide library (combinatorial library, etc.).

The method for bringing the test substance into contact with the cells is not particularly limited. For example, in exemplary method, a test substance may be placed into a vessel of a cell culture to be cultured therein, or a test substance may be mixed with cells.

A test substance showing RARRES3 gene expression can be selected as an inhibitor of RNA virus replication. The inhibitor selected as such can be used as a pharmaceutical antiviral drug or as a virus replication inhibitor.

### EXAMPLES

Hereinafter, the present invention will be described more specifically by means of examples. The scope of the present invention, however, should not be limited to these examples.

### Example 1

1. Methods

Cells

As previously described^{31,32}, mycoplasma-free human hepatoma cell line Huh-7.5 and PH5CH8 immortalized human hepatocytes were cultured in DMEM-high glucose supplemented with 10% fetal bovine serum, 1× penicillin-streptomycin, 1×GlutaMAX-I and 1×MEM non-essential amino acid solution (Thermo Fisher Scientific).

Liver tissues for obtaining fetal hepatocytes were provided by Advanced Bioscience Resources, certified non-profit corporate foundation. The tissues were collected with written informed consent from all donors pursuant to the Good Tissue Practice regulations, U.S. Food and Drug Administration's Code of Federal Regulations, Part 1271. Tissue processing, and isolation and culture of the hepatoblasts were carried out as previously described³². Use of the purchased fetal hepatocytes was determined to be exempt from review by the Institutional Review Board at the University of North Carolina (UNC) at Chapel Hill.

HAV Challenge in Genetically Modified Mice

Mice were bread and raised at the UNC-Chapel Hill according to the policies and guidelines of the institutional animal care and use committee. C57BL/6, Ifnar1^{-/-}, Irf3^{-/-} and Irf1^{-/-} mice were purchased from The Jackson Laboratory. As previously described⁷, 6-10-week-old mice were intravenously inoculated with hepatitis A virus. The mice were housed in individual cages and fecal pellets and serum samples were collected on regular basis. Tissues were collected upon necropsy, and stored in RNAlater (Thermo Fisher Scientific) or snap frozen on dry ice to be preserved at −80° C. until RNA extraction treatment. All experiments employing the mice were approved by the UNC-Chapel Hill Institutional Animal Care and Use Committee.

Reagents and Antibodies

MicroRNA-122 mimics were synthesized by Dharmacon and transfected as miRNA/miRNA* duplex by electroporation as previously described³³. Puromycin, Blasticidin and Ruxolitinib were purchased from InvivoGen. Pyridone 6 was obtained from EMD Millipore. Recombinant human IFN-λ1 and IFN-α and actinomycin D were purchased from Sigma-Aldrich. Recombinant human IFN-γ was obtained from PeproTech. PSI-7977 (sofosbuvir) was obtained from ChemScene and 2′-C-methyl adenosine (2′ CMA) was obtained from Santa Cruz Biotechnology. Cell viabilities were determined using Cell Counting Kit-8 (Dojindo) on 96-well plates according to the manufacturer's protocol.

Reagents used and suppliers thereof were as follows.

The followings were obtained from Cell Signaling Technology:

IRF-1 (D5E4)XP (1:500 dilution, catalog no. 8478);

IRF-7 (D2A1J) (1:500 dilution, catalog no. 13014);

IFIT1 (1:500 dilution, catalog no. 14769);

Stat1 (D1K9Y) (1:500 dilution, catalog no. 14994);

STING (D2P2F) (1:500 dilution, catalog no. 13647);

MyD88 (D80F5) (1:500 dilution, catalog no. 4283);

TLR3 (D10F10) (1:500 dilution, catalog no. 6961);

NF-κB p65 (D14E12) XP (1:500 dilution, catalog no. 8242);

mTOR (7C10) (1:500 dilution, catalog no. 2983);

Phospho-mTOR (Ser 2448) (D9C2), catalog no. 5536);

Phospho-mTOR (Ser 2481) (1:500 dilution, catalog no. 2974);

p70 S6 kinase (1:1,000 dilution, catalog no. 2708);

Phospho-p70 S6 kinase (Thr 389) (1:1,000 dilution, catalog no. 9234);

4E-BP1 (1:1,000 dilution, catalog no. 9444); and

Phospho-4E-BP1 (Thr 70) (1:1,000 dilution, catalog no. 9455).

IRF-3 (FL-425) (1:200 dilution, catalog no. sc-9082) and 2′-5′-oligoadenylate synthase 1 (OAS1) (1:100 dilution, catalog no. sc-374656) were obtained from Santa Cruz Biotechnology.

Anti-DHX58/RLR (1:500, catalog no. ab67270) was obtained from Abcam.

GAPDH monoclonal antibodies were obtained from Thermo Fisher Scientific (Clone 6C5; 1:10,000 dilution, catalog no. AM4300) or Wako (Clone 5A12; 1:4,000 dilution, catalog no. 016-25523).

RIG-I (Clone Alme-1; 1:1,000 dilution, catalog no. ALX-804-849) and Cardif (VISA/IPS-1/MAVS; 1:2,000 dilution, catalog no. ALX-210-929) were obtained from Enzo Life Sciences.

Anti-beta actin (Clone AC-74; 1:40,000 dilution, catalog no. A2228), anti-a tubulin (Clone DM1A; 1:15,000 dilution, catalog no. T6199) and anti-IL28RA (IFNLR1; 1:500 dilution, catalog no. AV48070) were obtained from Sigma-Aldrich.

IFNAR1 (1:2,000 dilution, catalog no. A304-290A) and NMI (1:4,000 dilution, catalog no. A300-551A) were obtained from Bethyl Laboratories, LMP2 (PSMB9; 1:400 dilution, catalog no. 14544-1-AP), APOL1 (1:500 dilution, catalog no. 11486-2-AP) and RARRES3 (1:800 dilution, catalog no. 12065-1-AP) were obtained from Proteintech.

IRDye 680 or 800 secondary antibodies (including catalog nos. 926-32211, 926-32212, 926-32214, 926-68020 and 926-68073 (1:12,000)) were purchased from LI-COR Biosciences.

Viruses

High-titer HAV (HM175/18f strain) was mycoplasma-free and was prepared as previously described³⁴. HAV infection was performed at a multiplicity of infection (MOI) of 10. SeV (Cantell strain) was obtained from Charles River Laboratories, and was inoculated at 50 U ml⁻¹ unless otherwise indicated. Infection with HCV-carrying Gaussia luciferase (GLuc) reporter was performed as previously described³². DENV serotype 2 (olSa-054 strain) and ZIKV (MR-766 and AB-59 strains) were propagated in Vero, C6/36 or Huh-7.5 cells and inoculated at MOI=1 as previously described³⁵.

HM175/18f-NLuc Reporter Virus

pHM175/18f-NLuc plasmid was prepared by PCR amplification of the NLuc open reading frame using pNL1.1 plasmid (Promega) as a template and primers containing a triglycine sequence flanked by XbaI and BamHI restriction enzyme sites. This PCR product was digested with these enzymes and ligated into similarly digested pSK-2A-Zeo-2B plasmid³⁶ to give pSK-2A-NLuc-2B plasmid. This plasmid was further digested with SacI/PflMI to cleave out the entire 2A-NLuc-2B fragment, which was ligated into similarly digested HM175/18f parental plasmid³⁷ to give pHM175/18f-NLuc reporter virus.

DENV/NLuc Reporter Virus

Plasmids encoding capsid and subgenomic RNA containing NS1-5 regions fused with a NanoLuc reporter flanked by 5′ and 3′ untranslated RNAs derived from DENV1 (D1/Hu/Saitama/NIID100/2014 strain) and DENV2-derived premembrane and envelope protein (olSa-054 strain) were transfected into HEK293T cells. Infectious virons secreted in the supernatant were harvested according to the previously described method³⁸.

Other Plasmids

pJFH1-QL containing cell culture-adaptive mutation Q221L in NS3 helicase, pJFH1/GND, pH77S.3, pH77D, pT7-18f, pHAV-Luc and pHAV-LucA3D are previously described^{32,34,39}. Lentiviral transfer plasmids encoding IRF1 effector genes (PLAAT4/RARRES3, PSMB9 and APOL1) were prepared by amplifying host genes by PCR using complementary DNA synthesized using total RNA derived from PH5CH8 cells as a template and primers having XbaI and PstI or NheI restriction enzyme sites. The PCR products were digested with these enzymes, and ligated into similarly digested pCSII-EF-MCSII plasmid to obtain pCSII-EF-RARRES3, -PSMB9 and -APOL1. Apoint mutation in pCSII-EF-RARRES3/C113S was introduced by primer site-directed mutagenesis of the sequence spanning the XbaI and PstI sites. The firefly luciferase reporter vectors including pIFN-O-Luc and p4×PRDIII-I-Luc, and Renilla luciferase control reporter vector pRL-TK are previously described^{3,31}.

Transcription and Transfection of Viral RNA

In vitro transcription of HAV or HCV RNA was performed with T7 RiboMAX Express Large Scale RNA production system (Promega) according to the manufacturer's protocol. Transfection of viral RNA was carried out in Gene Pulser Xcell Total System (Bio-Rad Laboratories) as previously described³³, or with TransIT-mRNAtransfection kit (Mirus) for HAV-Luc RNA as previously described³².

Production and Transduction of Lentiviruses

For production of shRNA lentiviruses, shRNA plasmid obtained from Sigma-Aldrich was co-transfected into 293FT cells with MISSION Lentiviral Packaging Mix (catalog no. SHP001; Sigma-Aldrich)). Supernatants collected 48 and 72 hours after the transfection were filtered through a 0.22-μm syringe filter. sgRNA CRISPR-Cas9a lentivirus was produced by co-transfecting the sgRNA expression vectors listed in 3rd Generation Packaging System Mix (catalog no. LV053; abm). Infection with lentiviruses was performed with addition of 8 pg ml-1 polybrene, followed by antibiotic selection with 6 μg ml⁻¹ puromycin for single knockout cells or 6 μg ml⁻¹ puromycin plus 5 μg ml⁻¹ blasticidin for double knockout cells. In order to avoid cloning bias, antibiotic-resistant bulk cell populations were used for the experiments.

RNA Extraction and RT-gPCR

Total RNA extraction was performed using RNeasy Mini Kit (QIAGEN), QIAamp viral RNA Mini Kit (QIAGEN) or TRIzol (Thermo Fisher). HAV genomic RNA was detected by a two-step RT-qPCR analysis using SuperScript III First-Strand Synthesis System (Thermo Fisher Scientific) and iTaq Universal SYBR Green Supermix (Bio-Rad Laboratories), or Thunderbird SYBR qPCR Mix (TOYOBO). 5′-GGTAGGCTACGGGTGAAAC-3′ (SEQ ID NO:3) and 5′-AACAACTCACCAATATCCGC-3′ (SEQ ID NO:4) were used as HAV-specific primers

HCV RNA abundance were determined according to the previously described method³². IRF target genes were quantified using the primer pairs listed in Table 1.

TABLE 1

| | | SEQ | | SEQ |
|---|---|---|---|---|
| | | ID | | ID |
| Gene | Forward | NO | Reverse | NO |
| RARRES3 | GATTTTCCGCCTTGGCTATG | 5 | TTGCTCAGGACTGAGAAGAC | 6 |
| PSMB9 | GTGGATGCAGCATATAAGCC | 7 | AGTGACCAGGTAGATGACAC | 8 |
| APOL6 | CTATTGCTCCCAGGCTACGCA | 9 | CCCTGCAAGCTCCATTCGTAGT | 10 |
| GBP3 | CGCACAGGAAAATCCTACCT | 11 | ACACACCACATCCAGATTCC | 12 |
| ERAP2 | GGGCCTCATTACATATAGGGA | 13 | ATTCCATTGTGACCAGGTTG | 14 |
| APOL1 | ATAATGAGGCCTGGAACGGA | 15 | GGTTGTCCAGAGCTTTACGG | 16 |
| SAMD9L | AAGCTCTGAGAGCAGATAGG | 17 | TTGAGTTTTGCTGCAGTAGG | 18 |
| UBA7 | TGATGCCCTCGATTGTCTTC | 19 | ACTTTGAGCAGCTCACAACC | 20 |
| IFIT3 | CTGGCAATTGCGATGTACCA | 21 | GTTTCAGGCCCAAGAGAACC | 22 |
| CXCL8 | AAGAGCCAGGAAGAAACCAC | 23 | CTTGGCAAAACTGCACCTTC | 24 |
| NMI | GGAGTTACAAGAGGCTACCA | 25 | CGAGCTCACTTGAAACGAAC | 26 |
| TLR3 | TAGCAAACACAAGCATTCGG | 27 | AGGAATCGTTACCAACCACA | 28 |
| CFB | TTCCCTGACAGAGACCATAG | 29 | CTGTCTGATCCATCTAGCAC | 30 |
| TAP2 | CCTCACTATTCTGGTCGTGT | 31 | GATCCGCAAGTTGATTCGAG | 32 |
| IFIT2 | GAGAATTGCACTGCAACCATGAG | 33 | CGATTCTGAAACTCAGTCCGGTAA | 34 |
| APOL3 | ATCCACACAGCTCAGAACAG | 35 | CAGCAAATGCCAAGACCAAC | 36 |
| MX1 | CAGTTACCAGGACTACGAGA | 37 | GGGTGATTAGCTCATGACTG | 38 |
| DDX60 | CTTCTATCTGGTTGAACGCT | 39 | CAGGGAAGTTGAAATACGCA | 40 |
| ZNF827 | AATCGGGCGAGAGAAAACCGAA | 41 | GACAGTTGAAAGAGGAGCTCGGAA | 42 |
| CXCL1 | ATTCACCCCAAGAACATCCA | 43 | CAGGATTGAGGCAAGCTTTC | 44 |
| FYN | CAATGAGTACACAGCAAGAC | 45 | AGCTCTGTGAGTAAGATTCC | 46 |
| TENM3 | GACAGCTCCAAACAGTTTACCTCA | 47 | TGTCTCGCAGGTCATAGCGAA | 48 |
| COL4A1 | GCCTGGTGAGTTTTATTTCGAC | 49 | ACGCTCTCCTTTCAATCCTAC | 50 |
| COL4A2 | GGTTTCTACGGAGTTAAGGG | 51 | TTCACCCTTGTACTGATCTG | 52 |
| DPYSL3 | GAGCAAACCCGCATGTTGGA | 53 | GCAATGGTGATGGCACGGAA | 54 |
| ACTB | GACCCAGATCATGTTTGAGACC | 55 | GTCACCGGAGTCCATCACGA | 56 |

Primer pair targeting DENV genomic RNA, 5′-ACCAGATCATCATTACAGGA-3′ (SEQ ID NO:57) and 5′-CATCATTAAGTCGAGGGCC-3′ (SEQ ID NO:58), or primer pair targeting ZIKV genomic RNA, 5′-AARTACACAACAACAAAGTGTGT-3′ (SEQ ID NO:59) and 5′TCCRCTCCYCTYCTYCTGTGTCTG-3′ (SEQ ID NO:60) was used with RNA-direct SYBR Green Realtime PCR Master Mix (TOYOBO) to quantify DENV and ZIKV RNA abundance.

Preparation for Phospholipid Analysis

Comprehensive analysis of phospholipid was as previously described^{4,41}. Briefly, total phospholipid was extracted from cell cultures by employing the Bligh-Dyer method.

An aliquot of lower organic phase was evaporated to dryness under N₂, and then the residue was dissolved in methanol for quantification of phosphatidyl choline and phosphatidyl ethanolamine by liquid chromatography-tandem mass spectrometry (LC/MS/MS). In order to analyze phosphatidic acid, phosphatidyl serine, phosphatidyl inositol, PI phosphate, bis-phosphoric acid and tris-phosphoric acid, an equal volume of methanol was added to another aliquot of the same lipid extract and the resultant was loaded onto a diethyl aminoethyl cellulose column (Santa Cruz Biotechnology) that was pre-equilibrated with chloroform. Following successive washes with chloroform/methanol (1:1, v/v), acidic phospholipid was eluted with chloroform/methanol/HCl/water (12:12:1:1, v/v) and then evaporated to dryness to give a residue, which was dissolved in methanol. The resulting fraction was subjected to methylation with trimethylsilyldiazomethane before LC/MS/MS analysis⁴².

Mass Spectrometry

LC-electrospray ionization-MS/MS analysis was performed using UltiMate 3000 LC System (Thermo Fisher Scientific) equipped with HTC PAL autosampler (CTC Analytics). 10 μl of the lipid sample was injected to separate the lipids on Waters X Bridge C18 column (3.5 μm, 150 mm×1.0 mm inner diameter) at room temperature (25° C.) using the following gradient solvent system: mobile phase A (isopropanol/methanol/water (5:1:4, v/v/v) supplemented with 5 mM ammonium formate and 0.05% ammonium hydroxide)/mobile phase B (isopropanol supplemented with 5 mM ammonium formate and 0.05% ammonium hydroxide) at ratios of 70/30% (0 min.), 50/50% (2 min.), 20/80% (13 min.), 5/95% (15-30 min.), 95/5% (31-35 min.) and 70/30% (35-45 min.). Flow rate was 20 μl/min.

Selected reaction monitoring were performed by running the triple quadrupole mass spectrometer (TSQ Vantage AM; Thermo Fisher Scientific) in positive ion mode to determine the phospholipid species. The characteristic fragments of individual phospholipids were detected by product ion scan (MS/MS mode). The chromatography peak areas were used for comparative quantification of each molecular species (for example, 38:6, 40:6) in a given class of phospholipids (for example, phosphatidic acid, phosphatidyl choline).

Immunoblotting

Western blotting was performed by standard methods. Odyssey CLx Infrared Imaging System (LI-COR Biosciences) was used for imaging.

RNA Interference

The siRNA pools listed in Table 2 were obtained from Dharmacon or Thermo Fisher Scientific, and were transfected into cells using siLentFect Lipid Reagent for RNAi (Bio-Rad Laboratories) or Lipofectamine RNAiMAX Transfection Reagent (Thermo Fisher Scientific) according to the manufacturer's protocol.

TABLE 2

| | Gene | | | | |
|---|---|---|---|---|---|
| Gene Symbol | Accession | Sequence (SEQ ID NO) | | | |
| RARRES3 | NM_004585 | GCACUGGGCCCUGUAU | 61 | UAUGGCAAGUCCCGCU | 62 |
| | | CAACAGUGCAGAGGUG | 63 | CGAAGGAGAUGGUUGG | 64 |
| PSMB9 | NM_148954 | GCAAAUGUGGUGAGAA | 65 | GAACCGAGUGUUUGAC | 66 |
| | | GGCAGCACCUUUAUCU | 67 | ACGUGAAGGAGGUCAG | 68 |
| GBP3 | NM_018284 | GAGAAGACCCUCACUA | 69 | CCACUGAAGUCUAUAU | 70 |
| | | GAACAGGCCCGAGUAC | 71 | CGCAUAAGCUAAAGAUC | 72 |
| SAMD9L | NM_152703 | GGAAGGGUCUAAACAG | 73 | GUAGGAGCAUUACUGU | 74 |
| | | GCAACGGGAUGUAGAU | 75 | CAGAAAAGGAUUUGCG | 76 |
| IFIT3 | NM_001549 | GCAAUAUGCUAUGGAC | 77 | GACUGGCAAUUGCGAU | 78 |
| | | GAGACGGAAUGUUAUC | 79 | UAGAGUGUGUAACCAG | 80 |
| APOL6 | NM_030641 | GAGAGAAUUUCCCAGA | 81 | AGAAACACCUUGAAGUA | 82 |
| | | GAACAACACUGGCGAU | 83 | GGGAAGUGGGAGUCGA | 84 |
| ERAP2 | NM_022350 | GAAAGCUGCUGAACUC | 85 | GAUCAUCUCUGGCACA | 86 |
| | | GAGUAGGUCUGAUUCA | 87 | GAUCACAUCUGGAUAU | 88 |
| UBA7 | NM_003335 | GAACAAAGCCCUGGAA | 89 | GGGCAGUGCUACAGUA | 90 |
| | | GCACUUCCCACCUAAUA | 91 | UGAAGCCUCUGAUGUU | 92 |
| APOL1 | NM_003661 | GUUCCAAGUGGGACAG | 93 | ACGAUAAAGGCCAGCA | 94 |
| | | AGAAUAUAUUGACGGAA | 95 | AAUGGGAACUGGAGAG | 96 |
| CFB | NM_001710 | CGAAGCAGCUCAAUGA | 97 | GGAGAUAGAAGUAGUC | 98 |
| | | ACACGUACCUGCAGAU | 99 | ACAGGAAGGGUACCGA | 100 |
| APOL3 | NM_145640 | GGUCAAGCAGAGAGAA | 101 | CAACCUUGUAUACGAG | 102 |
| | | CAACCAGCAUUGACCG | 103 | CCUGUGACCACCUGGC | 104 |
| NMI | NM_004688 | CCAAAGAAUUCCAGAUU | 105 | GCUCGAAAGUUCCUUA | 106 |
| | | CAAGUGAGCUCGAAAG | 107 | CGAAAGUUCCUUAUGA | 108 |
| MX1 | NM_002462 | UCACAGAUGUUUCGAU | 109 | GAAUGGGAAUCAGUCA | 110 |
| | | CCACAAAUGGAGUACAA | 111 | CGACAUACCGGAAGAC | 112 |
| TLR3 | NM_003265 | GAAGCUAUGUUUGGAA | 113 | GAAGAGGAAUGUUUAAA | 114 |
| | | GAUCAUCGAUUUAGGA | 115 | CAACAUAGCCAACAUAA | 116 |
| IFIT2 | NM_001547 | CAAAUUGGGUGCUGCU | 117 | GGAGAAAGCCCCAGGU | 118 |
| | | GCAAAAGUCUUCCAAG | 119 | GAACUAAUAGGACACGC | 120 |
| TAP2 | NM_018833 | GUAACUGGCUUCCUUU | 121 | CAUGAAGUCUGUCGCU | 122 |
| | | GGAAAUGGAGCAUGGA | 123 | GAAACAACGUCUGGCC | 124 |
| DDX60 | NM_017631 | GAAGGUAUUUGGUCGA | 125 | GCACUCACCAUUAAAUC | 126 |
| | | GGAGAGAGGUAUAAUG | 127 | AAAUGUCGCUUAAUGC | 128 |
| RPS6KB1 | NM_003161 | GGACUAUGCAAAGAAU | 129 | GGUUUUUCAAGUACGA | 130 |
| IRF7 | NM_001572 | GUCUAAUGAGAACUCC | 131 | GCCUAGAACCCAGUCU | 132 |
| IRF1 | NM_002198 | UCACAGAUCUGAAGAAC | 133 | CCAAGAACCAGAGAAAA | 134 |
| Non-targeting | N/A | UAGCGACUAAACACAUC | 135 | AUGUAUUGGCCUGUAU | 136 |
| Control | | UAAGGCUAUGAAGAGA | 137 | AUGAACGUGAAUUGCU | 138 |

Luciferase Assay

GLuc analysis of HCV replication and dual luciferase assay for analyzing the transcriptional induction were carried out according to the previously described methods^{31,32}. NanoLuc activity was determined with Nano-Glo Luciferase Assay System (Promega) according to the manufacture's protocol. For virus replication assays, the medium was replaced with a drug-containing medium at 1 hour post inoculation.

RNA Sequence (RNA-Seq) Analysis

RNA purity was measured with NanoDrop 2000 spectrophotometer (Thermo Fisher Scientific), and integrity was assessed with 2100 Bioanalyzer Instrument (Agilent Technologies). Qualities of RNA and sequence analysis were comparable for all samples. Sequencing was performed on HiSeq 2000 platform (Illumina). RNA sequences were aligned with hg38 using STAR v.2.4.2a⁴³, sequences were quantified using SalmonBeta-0.4.2⁴⁴, and difference in the expression levels between the samples was determined using DESeq2⁴⁵. Gene ontology analysis was performed with DAVID 6.8.

Confocal Laser Scanning Microscope

Cells grown on an 8-well chamber slide (Falcon) were fixed with 4% paraformaldehyde and permeabilized with 0.25% Triton X-100. Subsequently, the cell monolayer was incubated with rabbit anti-IRF-1 antibody (1:50 dilution, catalog no. 8478; Cell Signaling Technology) at 4° C. overnight, followed by a secondary antibody and goat anti-rabbit Alexa Fluor 488 (1:200 dilution, Thermo Fisher Scientific). Nuclei were counterstained with 4,6-diamidino-2-phenylindole (DAPI). Images were acquired using Leica DMIRB Inverted Microscope from UNC Michael Hooker Microscopy Facility.

Statistical Analysis

Unless otherwise specified, comparisons between the groups were all performed by analysis of variance (ANOVA) or Student's t-test using Prism 6.0 software (GraphPad Software). Unless otherwise indicated, p values were calculated from three biological replicates. In some of the experiments for validating earlier conclusions using orthogonal approaches, two independent experiments each with three technical replicates were conducted. These few exceptions are noted in the figure legends.

Results and Discussion

The current models of cell-intrinsic immunity against RNA virus center on virus-triggered antiviral response initiated by RIG-I-like receptors or Toll-like receptors which sense pathogen-associated molecular patterns, and downstream signaling through interferon regulatory factors (IRFs) serving as transcription factors that induce synthesis of type-I and type-III interferonst. RNA viruses developed high-level strategies to inhibit these signaling pathways and avoid elimination by cells, which proves the importance of these signals². Meanwhile, not much attention has been paid as to how IRFs are maintaining the baseline level of protection mechanism against the viruses.

In this example, a set of antiviral factors that were supposed to have relation to RIG-I-like receptor and Toll-like receptor signalings were knocked down to map critical host pathways that restrict positive-sense RNA virus replication in immortalized hepatocytes, and as a result, unexpected roles of IRF1 were identified. Constitutively expressed IRF1 acts independently of mitochondrial antiviral signaling (MAVS) protein, IRF3 and signal-transducer-and-activator-of-transcription-1 (STAT1)-dependent signaling, showing that it provides an intrinsic antiviral protection function in actinomycin D-treated cells.

IRF1 was found to localize to the nucleus, where it maintained baseline level transcriptions of a group of antiviral genes that were involved in the protection against infection with diverse pathogenic RNA viruses including hepatitis A and C viruses, dengue virus and Zika virus. The findings by the present inventors not only revealed the existence of a previously unrecognized protection layer as an immune mechanism intrinsic in the hepatocytes against these positive-sense RNA viruses, but also resulted in identifying a number of IRF1 effector genes that have been unknown to have antiviral functions.

In order to reveal the host antiviral pathways, immortalized adult human hepatocytes were analyzed.

Similar to hepatocytes in vivo, PH5CH8 cells express RIG-I-like receptors (RLRs) and Toll-like receptors (TLRs), and induce potent interferon (IFN) and proinflammatory cytokine responses upon infection with RNA viruses³⁻⁵. The present inventors depleted expressions of RLR- and TLR-related antiviral factors by transducing cells with lentiviral vectors expressing short-hairpin RNAs (shRNAs), and assessed the influence on replication of hepatitis A virus (HAV) which is a hepatotropic human picornavirus that causes an acute inflammatory liver disease⁶.

Surprisingly, while depletion of the following RLRs, signaling adaptors, transcription factors and IFN receptors resulted in only a small increase in HAV replication, depletion of IRF1 increased the HAV RNA abundance 30-fold (Figure Ta).
RLRs involved in inducible IFN responses: retinoic acid-inducible gene I protein (RIG-I), melanoma differentiation-associated protein 5 (MDA5), and ATP-dependent RNA helicase LGP2
Signal transduction adaptors: mitochondrial antiviral signaling (MAVS) proteins, stimulator of interferon genes (STING), myeloid differentiation primary response protein (MyD88), and TIR-domain-containing adaptor-inducing interferon-3 (TRIF)
Transcription factors: interferon regulatory factor 3 (IRF3) and IRF7
IFN receptors: interferon α/β receptor 1 (IFNAR1) and IFNλ receptor 1(IFNLR1)

Significant increase in HAV replication resulting from IRF1 depletion was confirmed in CRISPR-Cas9-introduced PH5CH8 knockout cell pools expressing different single guide RNAs (sgRNAs) (IRF1 no. 1 and IRF1 no. 2) (Figure Tb). In contrast, IRF3 knockout or depletion of both IRF3 and IRF7 had little effect on replication (FIG. 1b).

Accordingly, IRF1 was found to be significantly more active than IRF3 in suppressing HAV infection in these cells. Furthermore, while genetically-deficient Irf1^{-/-} mice shed more HAV in the feces and had significantly more viral RNA in the liver than Irf3^{-/-} or wild-type mice 7 days after virus challenge, HAV did not establish persistent infection like that in Ifnar1^{-/-} mice⁷ (FIG. 1c). IRF1 has functions of promoting IFN-γ signaling, major histocompatibility complex class I expression and T cell activation in vivo^{8,9}. Since, however, neither IFN-γ receptor knockout nor depletion of functional T cells permit infection of C57BL/6 mice⁷, these actions of IRF1 on the immune cells are unlikely to be involved in the enhancement of HAV replication in Irf1^{-/-} mice.

These results suggest that IRF1 suppresses virus replication in hepatocytes.

IRF1 is known to induce type-I IFN gene expression, mediate type-III IFN expression downstream of MAVS protein localized on peroxisome¹¹, and exert broad antiviral effector activity.¹² When, however, receptors for type-I or type-III IFN (IFNAR1 and IFNLR1) were knocked out, HAV infection was enhanced by less than 3-fold (FIG. 1d). Moreover, no increase in HAV replication was caused when signal-transducer-and-activator-of-transcription-1 (STAT1) was knocked out such that both type I and type III IFN signalings were neutralized (FIG. 1e, left panel) but replication increased by 20-fold or more when IRF1 was additionally knocked out (FIG. 1e, right panel).

Similarly, pharmacological inhibition of Janus kinases (Jak-1/2) which are important components for IFN-induced Jak/STAT signaling enhanced replication by 2-fold but showed no effect of attenuating the increase in replication caused by IRF1 knockout (FIG. 1f). In brief, these data show that IRF1 restricts HAV replication independent of IFN signaling.

IRF1 expression is known to result from transcriptional induction that is dependent on transcription factor NF-κB via RLR signal-dependent activation of adaptor protein MAVS^{5,11,13}. However, knocking out NF-κB subunit RelA did not enhance HAV replication (Figure Tb), and increases resulting from IRF1 knockout were not lessened in MAVS (or IRF3)-knockout cells (FIG. 1g). Furthermore, IRF1 knockout did not reduce Sendai virus (SeV)-induced IFN-β promoter activity or IFN-stimulated gene (ISG) expression, but these RIG-I-dependent responses were suppressed in IRF3-knockout cells. Similarly, while an antiviral response triggered by MAVS overexpression was dependent on IRF3, it did not require IRF1. Hence, IRF1 suppresses HAV infection independently of RelA and MAVS signaling.

Accordingly, while only IRF1 knockout enhanced infection with HAV infectious particles (Figure Tb), replication of synthetic HAV RNA transfected by electroporation was enhanced not only in IRF1-knockout cells but also in IRF3-knockout cells. IRF1 and IRF3 knockouts resulted equivalent and additive increases up to 3 days post-transfection, IRF1-knockouts (both IRF1-sgRNAs no. 1 and no. 2) showed a greater effect on Day 5 when continuous de novo virus replication manifests.

Induction of IRF3-dependent ISG expression was observed by electroporation of RNA into cells, which was not observed with infection with virus particles. This presumably demonstrates that transfection by electroporation instantly allows a larger amount of virus RNA to be loaded into the cytoplasm as compared to infection with virus particles. In summary, these results show that IRF1 and IRF3 act independently, and that IRF1 mediates protection against HAV infection that does not elicit IRF3-dependent response, at an early post-entry stage.

Inhibition of IRF1 expression also promoted replication of HAV as well as replication of hepatitis C virus (HCV), dengue virus (DENV) and Zika virus (ZIKV) belonging to the family Flaviviridae in human hepatoma cell line Huh-7.5 cells deficient in RIG-I and TLR3 signalings^{3,14} (FIGS. 1h-1j). Since enhancement of HCV, HAV or DENV replication was not seen in the Huh-7.5 cells by ruxolitinib treatment, lack of IFN response was confirmed.

Thus, IRF1 suppresses replication of multiple pathogenic positive-sense RNA viruses in hepatocyte-derived cells. Also in PH5CH8 cells, HCV RNA replication was more enhanced by knocking down IRF1 expression than by knocking down IRF3, RLR, MAVS or IFN receptor. As in the case of HAV, HCV suppression by IRF1 was not reduced by pharmacological blockade of IFN signaling.

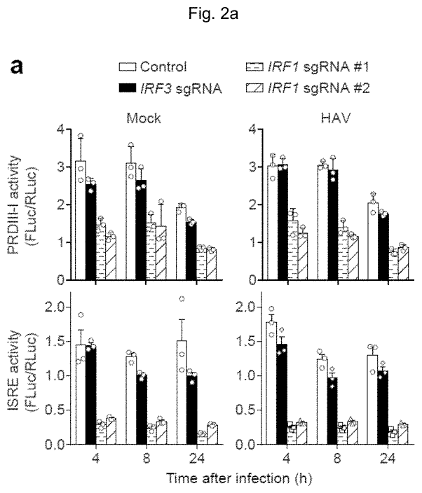

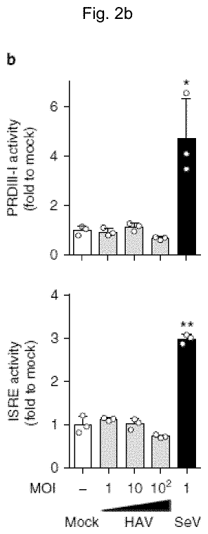

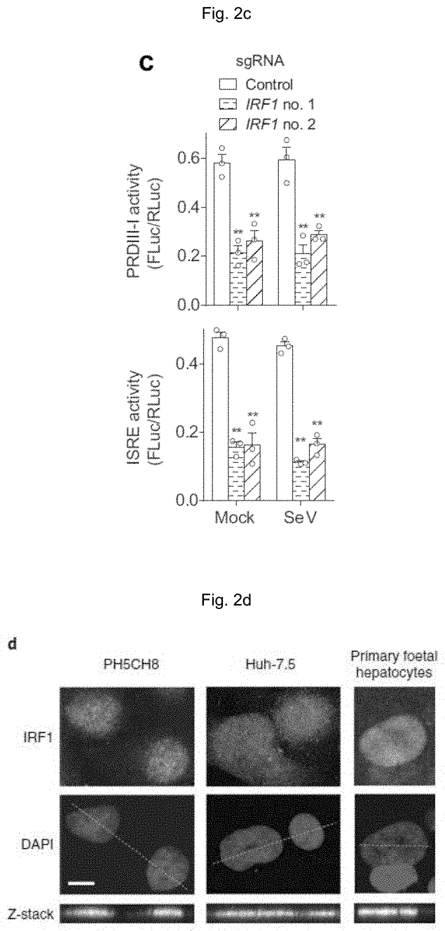

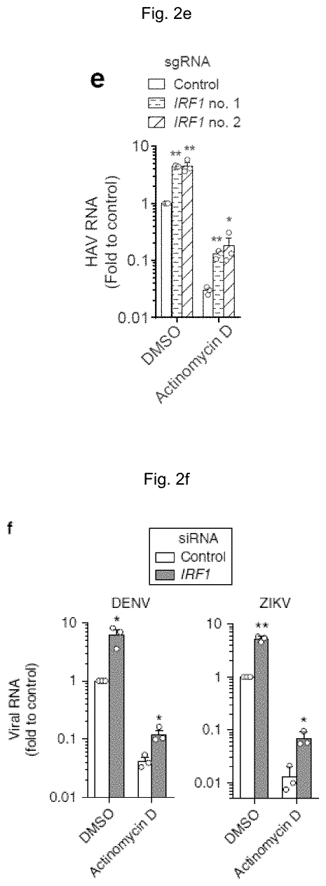

IRF1 protein abundance was not increased in HAV-infected PH5CH8 cells, and high multiplicity infection by HAV did not enhance the activities of IRF1-responsive PRDIII-I and IFN-stimulated response element (ISRE) promoters (FIGS. 2a and 2b)¹⁵¹⁷. When, however, IRF1 was knocked out, baseline level activities of these promoters were notably reduced in both PH5CH8 cells and Huh-7.5 cells (FIGS. 2a and 2c), while they were not reduced with a Jak/STAT signaling inhibitor, ruxolitinib.

In summary, these results suggest that the baseline level expression of IRF1 provides intrinsic antiviral protection by maintaining constitutive transcription of the antiviral genes, which is consistent with constitutive nuclear localization of IRF1 in uninfected PH5CH8 and Huh-7.5 cells and primary human fetal hepatocytes (FIG. 2d). As a further support for this hypothesis, suppression of IRF1 expression promoted replications of HAV, DENV and ZIKV when synthesis of new mRNA was inhibited in actinomycin D-treated Huh-7.5 cells (FIGS. 2e and 2f).

In order to identify antiviral effectors that are regulated specific to IRF1, transcription profiles of HAV-infected IRF1- and IRF3-knockout PH5CH8 cells were compared (FIG. 3a). Compared to cells expressing control sgRNA, changes in the transcript abundance were highly congruent in the two independent IRF1 knockout cell lines, where 51 genes showed similar expression reduction of 2-fold or more in both cell lines (Spearman's r=0.814; FIGS. 3a and 3b).

Specifically, these genes included known viral sensors (IFIH1, TLR3), IFN-regulated antiviral effectors (MX1, IFIT2, IFIT3), chemotactic factors (CCL2, CXCL1, CXCL2, CXCL8) and components of immunoproteasome (proteasome subunit 08 (PSMB8), PSMB9 and PSMB10), as well as multiple genes that had not been recognized to have antiviral functions. Only three of these transcripts were downregulated by 2-fold or more in the IRF3-knockout cells (FIG. 3b).

The present inventors focused on the 18 genes that were most downregulated in the IRF1-knockout cells. Two-fold or more reduction in the base line expression was confirmed for each of the genes except CXCL8 in uninfected IRF1-knockout PH5CH8 cells by quantitative reverse transcription PCR (RT-qPCR) (FIGS. 3c and 3d). In the IRF1-knockout cells, reduction in the baseline level expressions of PSMB9, N-myc-interactor (NMI) and TLR3 protein were also observed, and poly(I.C) recognition by TLR3 disappeared as well.

Importantly, influence of IRF1 knockout on transcript levels was equivalent in HAV-infected cells and uninfected cells (Spearman r=0.944-0.963, P<0.001; FIG. 3c). Thus, IRF1 suppresses HAV replication by promoting constitutive baseline level transcription of the antiviral effector genes. Expressions of all of these genes were also confirmed in primary cultured human hepatocytes and hepatoblasts¹⁸.

When small interference RNA (siRNA) pools targeting phospholipase A and acyltransferase 4 (PLAAT4/RARRES3), apolipoprotein L6 (APOL6), endoplasmic reticulum aminopeptidase 2 (ERAP2), N-myc and STAT-interacting factor (NMI) or MX dynamin-like GTPase 1 (MX1) were transfected into PH5CH8 cells, HAV replications were all enhanced by 3-fold or more (FIG. 3e). From these results for all genes except APOL6, correlation was confirmed between knockdown efficiency of the individual siRNAs and replication enhancement.

Importantly, when expressions of PLAAT4/RARRES3, ERAP2, NMI and MX1 were knocked down simultaneously, replication was promoted by about 40-fold (FIG. 3f), recapitulating the phenotype of IRF1-knockout cells (FIG. 1b). In similar experiments using Huh-7.5 cells, different subsets of IRF-regulated genes were confirmed to restrict replication of HCV (PSMB9, APOL1 and MX1), and DENV and ZIKV (PSMB9 and MX1) (FIGS. 3g-3l). Antiviral activities of PSMB9 against HCV and the flaviviruses, and HCV-specific antiviral activity of apolipoprotein L1 were confirmed by overexpression.

Accordingly, IRF1 regulates baseline level expressions of a group of genes, which suppress replications of different positive-sense RNA viruses in various combinations. Knockdowns of these genes were confirmed to have no influence on cell proliferation.

PLAAT4/RARRES3, gene that was most downregulated by IRF1 knockout and most active in suppressing HAV replication (FIGS. 3e and 3f), encodes single-pass transmembrane protein having acyltransferase activity¹⁹. Although RARRES3 is previously shown to slightly suppress poliovirus replication¹², it is not recognized as an important restriction factor for any virus. IFN-γ induced accumulation of IRF1 in the nucleus and restricted HAV replication in an IRF1-dependent manner in Huh-7.5 cells, but suppressive effect of IFN-γ weakened by RARRES3 knockdown.

Furthermore, RARRES3 expression in IRF1-knockout cells suppressed HAV replication whereas Cys¹¹³-Ser mutant (C113S) lacking acyltransferase activity did not (FIG. 4a). Similar results were also obtained in Huh-7.5 cells (FIG. 4a). Although PLA2G16, a paralog of RARRES3 (52% amino acid identity), is a factor that promotes viral entry for some picornaviruses²⁰, RARRES3 inhibited neither entry nor genomic translation of nanoluciferase-expressing HAV (HM175/18f-NLuc, “HAV/NLuc”; FIG. 4b) while it suppressed replication of subgenomic RNA replicon (FIG. 4c). RARRES3-knockout Huh-7.5 cells showed enhanced replication of HAV/NLuc virus (FIG. 4d). While action of RARRES3 against HAV was strong, RARRES3 overexpression did not restrict replications of HCV, DENV or human rhinovirus 14 (HRV-14).

The acyltransferase activity of RARRES3 possibly have a pleiotropic influence on cell signaling pathways including PI3K/Akt/mTOR pathways^{21,22}. RARRES3 overexpression induced phosphorylation of p70-S6K^{Thr389} in an acyltransferase-dependent manner, and reduced mTOR function by catalyzing phosphorylation of mTOR at Ser2448^{23,24}, and reduced mTOR-dependent phosphorylation of 4E-BP1 at Thr70 (FIGS. 4e and 4f). Consistent with this, phosphorylation of both p70-S6K and mTOR were reduced in IRF1-knockout cells (FIG. 4g). While pharmacological inhibition of mTOR also inhibited HAV, it did not inhibit HCV or DENV replication (FIGS. 4h and 4i).

Accordingly, while other action mechanisms cannot be excluded, RARRES3 seems to exert an antiviral action via suppression of mTOR function. Although RARRES3 has phospholipase A activity¹⁹, only slight increase in phosphoinositide PI(3,4,5)P3 was observed in overexpressed cells and no change was seen among 211 lipid species.

These data demonstrates that RARRES3 is a key HAV suppression factor for IRF1 to control transcription.

Only MX1, among other three genes that have major suppression activity against HAV, has well known antiviral activity (FIGS. 3e and 3f). NMI has previously been suggested to promote degradation of IRF7, and to have a proviral function as a factor that negatively regulates IFN responses²⁵. While endoplasmic reticulum aminopeptidase 2 (ERAP2), an aminopeptidase, contributes to T cell responses by generating human leukocyte antigen class 1-binding peptides, its cell-intrinsic antiviral activity is unknown.

Interestingly, the present inventors found that PSMB9, a component of immunoproteasome that is also involved in antigen processing²⁶, provides baseline level antiviral protection against HCV and the flaviviruses, DENV and ZIKV (FIGS. 3g-3l). While further studies are needed, these results suggest the presence of an antiviral action by an antigen processing machinery that works in an unrecognized way, which may explain the reason why immunoproteasome is suppressed by many viruses^{26,27}. Although many of the genes whose baseline level expressions are regulated by IRF1 identified by the present inventors have previously been suggested of their involvement in IFN responses, only a few genes (for example, MX1 and IFIT3) have been confirmed to have direct antiviral function²⁸.

The data of the present inventors show that, among the genes that are regulated by IRF1 at baseline level, different combinations of genes have antiviral activities to different kinds of positive-sense RNA viruses (FIGS. 3e-3l). Differences are also considered to lie between mammal species, presumably reflecting the evolutionary process of the viruses. Because of the strong virus control by MAVS and IRF3/IRF7-mediated transcriptional responses⁷, mice (Mus musculus) are not permissive for HAV infection.

Nevertheless, even though PLAAT4/RARRES3 and ERAP2, orthologs of two of the four IRF-regulated genes that most strongly suppress HAV replication in human hepatocytes, did not exist in Irf1^{-/-} mice, enhancement of HAV replication was observed at an early stage after infection (FIG. 1c).

While IRF1 has been previously shown to contribute to baseline level expressions of tens of IFN-γ-inducible proinflammatory and antimicrobial genes in macrophages²⁹, functional importance of IRF1, which regulates gene expression at baseline level, in suppression of virus replication has not been recognized. Data of the present inventors show that constitutive expression of IRF1 in hepatocytes maintains baseline level transcription of a set of genes having unknown antiviral functions, thereby exerting early protection against viral entry. Since IRF1 also mediates early protection against alphaviruses in muscle cells independent of IFN³⁰, it may act similarly in non-hepatic tissues. Further elucidation of the action mechanism of the IRF1-regulated antiviral factors in suppressing virus replication may provide new directions for developing antiviral therapy targeting host factors.

All publications cited herein are incorporated by reference herein in their entirety. It will be apparent to those skilled in the art that the present invention is described with reference to certain preferable embodiments, however, various modifications and variations can be made in the invention and specific examples provided herein without departing from the spirit or scope of the invention. Thus, it is intended that the invention covers the modifications and variations of this invention that come within the scope of any claims and their equivalents.

### REFERENCES

1. Yoneyama, M., Onomoto, K., Jogi, M., Akaboshi, T. & Fujita, T. Viral RNA detection by RIG-I-like receptors. Curr Opin Immunol 32, 48-53 (2015).
2. Chan, Y. K. & Gack, M. U. Viral evasion of intracellular DNA and RNA sensing. Nat Rev Microbiol 14, 360-373 (2016).
3. Li, K., Chen, Z., Kato, N., Gale, M., Jr. & Lemon, S. M. Distinct poly(I-C) and virus-activated signaling pathways leading to interferon-beta production in hepatocytes. J Biol Chem 280, 16739-16747 (2005).
4. Woodson, S. E. & Holbrook, M. R. Infection of hepatocytes with 17-D vaccine-strain yellow fever virus induces a strong pro-inflammatory host response. J Gen Virol 92, 2262-2271 (2011).
5. Feng, H. et al. NLRX1 promotes immediate IRF1-directed antiviral responses by limiting dsRNA-activated translational inhibition mediated by PKR. Nat Immunol (2017).
6. Lemon, S. M., Ott, J. J., Van Damme, P. & Shouval, D. Type A viral hepatitis: A summary and update on the molecular virology, epidemiology, pathogenesis and prevention. J Hepatol (2017).
7. Hirai-Yuki, A. et al. MAVS-dependent host species range and pathogenicity of human hepatitis A virus. Science 353, 1541-1545 (2016).
8. Taki, S. et al. Multistage regulation of Th1-type immune responses by the transcription factor IRF-1. Immunity 6, 673-679 (1997).
9. White, L. C. et al. Regulation of LMP2 and TAP1 genes by IRF-1 explains the paucity of CD8+ T cells in IRF-1^{-/-} mice. Immunity 5, 365-376 (1996).
10. Fujita, T., Kimura, Y., Miyamoto, M., Barsoumian, E. L. & Taniguchi, T. Induction of endogenous IFN-alpha and IFN-beta genes by a regulatory transcription factor, IRF-1. Nature 337, 270-272 (1989).
11. Odendall, C. et al. Diverse intracellular pathogens activate type III interferon expression from peroxisomes. Nat Immunol 15, 717-726 (2014).
12. Schoggins, J. W. et al. A diverse range of gene products are effectors of the type I interferon antiviral response. Nature 472, 481-485 (2011).
13. Dixit, E. et al. Peroxisomes are signaling platforms for antiviral innate immunity. Cell 141, 668-681 (2010).
14. Sumpter, R., Jr. et al. Regulating intracellular antiviral defense and permissiveness to hepatitis C virus RNA replication through a cellular RNA helicase, RIG-I. J Virol 79, 2689-2699 (2005).
15. Leblanc, J. F., Cohen, L., Rodrigues, M. & Hiscott, J. Synergism between distinct enhanson domains in viral induction of the human beta interferon gene. Mol Cell Biol 10, 3987-3993 (1990).
16. Miyamoto, M. et al. Regulated expression of a gene encoding a nuclear factor, IRF-1, that specifically binds to IFN-beta gene regulatory elements. Cell 54, 903-913 (1988).
17. Tanaka, N., Kawakami, T. & Taniguchi, T. Recognition DNA sequences of interferon regulatory factor 1 (IRF-1) and IRF-2, regulators of cell growth and the interferon system. Mol Cell Biol 13, 4531-4538 (1993).
18. Oikawa, T. et al. Model of fibrolamellar hepatocellular carcinomas reveals striking enrichment in cancer stem cells. Nat Commun 6, 8070 (2015).
19. Uyama, T., Jin, X. H., Tsuboi, K., Tonai, T. & Ueda, N. Characterization of the human tumor suppressors TIG3 and HRASLS2 as phospholipid-metabolizing enzymes. Biochim Biophys Acta 1791, 1114-1124 (2009).
20. Staring, J. et al. PLA2G16 represents a switch between entry and clearance of Picomaviridae. Nature (2017).
21. Hsu, T. H. et al. Involvement of RARRES3 in the regulation of Wnt proteins acylation and signaling activities in human breast cancer cells. Cell Death Diff 22, 801-814 (2015).
22. Ou, C. C. et al. Downregulation of HER2 by RIG1 involves the PI3K/Akt pathway in ovarian cancer cells. Carcinogenesis 29, 299-306 (2008).
23. Chiang, G. G. & Abraham, R. T. Phosphorylation of mammalian target of rapamycin (mTOR) at Ser-2448 is mediated by p70S6 kinase. J Biol Chem 280, 25485-25490 (2005).
24. Figueiredo, V. C., Markworth, J. F. & Cameron-Smith, D. Considerations on mTOR regulation at serine 2448: implications for muscle metabolism studies. Cell Mol Life Sci 74, 2537-2545 (2017).
25. Wang, J. et al. Negative regulation of Nmi on virus-triggered type I IFN production by targeting IRF7. J Immunol (Baltimore, Md.: 1950) 191, 3393-3399 (2013).
26. McCarthy, M. K. & Weinberg, J. B. The immunoproteasome and viral infection: a complex regulator of inflammation. Frontiers in microbiology 6, 21 (2015).
27. Verweij, M. C. et al. Viral inhibition of the transporter associated with antigen processing (TAP): a striking example of functional convergent evolution. PLoS Pathog 11, e1004743 (2015).
28. Schoggins, J. W. et al. Pan-viral specificity of IFN-induced genes reveals new roles for cGAS in innate immunity. Nature 505, 691-695 (2014).
29. Langlais, D., Barreiro, L. B. & Gros, P. The macrophage IRF8/IRF1 regulome is required for protection against infections and is associated with chronic inflammation. J Exp Med 213, 585-603 (2016).
30. Nair, S., Poddar, S., Shimak, R. M. & Diamond, M. S. Interferon regulatory factor-1 (IRF-1) protects against chikungunya virus induced immunopathology by restricting infection in muscle cells. J Virol (2017).
31. Dansako, H. et al. Class A scavenger receptor 1 (MSR1) restricts hepatitis C virus replication by mediating toll-like receptor 3 recognition of viral RNAs produced in neighboring cells. PLoS Pathog 9, e1003345 (2013).
32. Yamane, D. et al. Regulation of the hepatitis C virus RNA replicase by endogenous lipid peroxidation. Nat Med 20, 927-935 (2014).
33. Yamane, D. et al. Differential hepatitis C virus RNA target site selection and host factor activities of naturally occurring miR-122 3′ variants. Nucleic Acids Res (2017).
34. Feng, Z. et al. A pathogenic picornavirus acquires an envelope by hijacking cellular membranes. Nature 496, 367-371 (2013).
35. Hishiki, T. et al. Interferon-mediated ISG15 conjugation restricts dengue virus 2 replication. Biochem Biophys Res Commun 448, 95-100 (2014).
36. Beard, M. R., Cohen, L., Lemon, S. M. & Martin, A. Characterization of recombinant hepatitis A virus genomes containing exogenous sequences at the 2A/2B junction. J Virol 75, 1414-1426 (2001).
37. Binn, L. N. et al. Primary isolation and serial passage of hepatitis A virus strains in primate cell cultures. J Clin Microbiol 20, 28-33 (1984).
38. Matsuda, M. et al. High-throughput neutralization assay for multiple flaviviuses based on single-round infectious particles using dengue virus type 1 reporter replicon. Sci Rep 8, 16624 (2018).
39. Yi, M. & Lemon, S. M. Replication of subgenomic hepatitis A virus RNAs expressing firefly luciferase is enhanced by mutations associated with adaptation of virus to growth in cultured cells. J Virol 76, 1171-1180 (2002).
40. Baba, T. et al. Phosphatidic acid (PA)-preferring phospholipase A1 regulates mitochondrial dynamics. J Biol Chem 289, 11497-11511 (2014).
41. Imae, R. et al. LYCAT, a homologue of C. elegans acl-8, acl-9, and acl-10, determines the fatty acid composition of phosphatidylinositol in mice. J Lipid Res 53, 335-347 (2012).
42. Kielkowska, A. et al. Anew approach to measuring phosphoinositides in cells by mass spectrometry. Adv Biol Regul 54, 131-141 (2014).
43. Dobin, A. et al. STAR: ultrafast universal RNA-seq aligner. Bioinformatics 29, 15-21 (2013).
44. Patro, R., Duggal, G., Love, M. I., Irizarry, R. A. & Kingsford, C. Salmon provides fast and bias-aware quantification of transcript expression. Nat Methods 14, 417-419 (2017).
45. Love, M. I., Huber, W. & Anders, S. Moderated estimation of fold change and dispersion for RNA-seq data with DESeq2. Genome Biol 15, 550 (2014).

### Example 2

Among the mTOR inhibitors, pictilisib that had the strongest virus replication suppression effect was used to validate the antiviral effect in infected mouse models. The method was as follows.

Virus: HAV-HM175 strain, 1.7×10⁹ GE, iv route

Vehicle: 0.5% methyl cellulose/0.2% Tween 80

Mice: Ifnar1^{-/-} C57BL/6, 5 animals/group

The drug was orally administered from Day 5 following the infection for 14 consecutive days except Day 9 and Day 16. Viral level in the feces was quantified with time because it can serve as an indicator that reflects the viral level in the liver.

As a result, the viral level was reduced to about one-tenth by administration of the drug, confirming its viral suppression effect (FIG. 6).

### SEQUENCE LISTING FREE TEXT

SEQ ID NOS:3-138: Synthetic nucleotides

Sequence listings: Patent application P25992020-072209_2.app

1. A method for treating a disease caused by an RNA virus in a subject, the method comprising administering an effective amount of a pharmaceutical composition comprising retinoic acid receptor responder protein 3 (RARRES3) and/or an mTOR inhibitor to a subject in need thereof.

2. The method of claim 1, wherein the mTOR inhibitor additionally has an activity of inhibiting phosphatidylinositol 3-kinase.

3. The method of claim 1, wherein the mTOR inhibitor is a rapamycin derivative or an mTOR complex inhibitor.

4. The method of claim 1, wherein the disease caused by an RNA virus is hepatitis A, herpangina, hand-foot-and-mouth disease, poliomyelitis or foot-and-mouth disease in swine.

5. A method of inhibiting RNA virus replication in a cell, the method comprising contacting the cell with a retinoic acid receptor responder protein 3 (RARRES3) and/or an mTOR inhibitor.

6. The method of claim 5, wherein the mTOR inhibitor is a dual inhibitor which additionally has an activity of inhibiting phosphatidylinositol 3-kinase.

7. The method of claim 5, wherein the mTOR inhibitor is a rapamycin derivative or an mTOR complex inhibitor.

8. The method of claim 5, wherein the RNA virus is hepatitis A virus, a coxsackievirus, an enterovirus, a poliovirus or a foot-and-mouth disease virus.

9. A method for screening an inhibitor of RNA virus replication, the method comprising:
providing a cell that is infected with an RNA virus;
bringing a test substance into contact with the cell;
measuring expression of retinoic acid receptor responder protein 3 (RARRES3) in the cell in the presence of the test compound;
comparing the expression of RARRES3 in the presence of the test compound with the expression of RARRES3 in the absence of the test compound; and
selecting a test compound that inhibits RARRES3 as an inhibitor of RNA virus replication.

10. A method for inhibiting RNA virus replication in a cell, the method comprising expressing a gene coding for retinoic acid receptor responder protein 3 in a cell.

11. The method of claim 9, wherein the RNA virus is hepatitis A virus, a coxsackievirus, an enterovirus, a poliovirus or a foot-and-mouth disease virus.

12. A pharmaceutical composition comprising a retinoic acid receptor responder protein 3 (RARRES3) and an mTOR inhibitor, and a pharmaceutically acceptable carrier.

13. The pharmaceutical composition of claim 12, wherein the mTOR inhibitor is a dual inhibitor which additionally has an activity of inhibiting phosphatidylinositol 3-kinase.

14. The pharmaceutical composition of claim 12, wherein the mTOR inhibitor is a rapamycin derivative or an mTOR complex inhibitor.

15. The pharmaceutical composition of claim 12, wherein the RNA virus is hepatitis A virus, a coxsackievirus, an enterovirus, a poliovirus or a foot-and-mouth disease virus.
